(19) United States
(12) Patent Application Publication
(10) Pub. No.: US 2025/0064967 A1
(43) Pub. Date: Feb. 27, 2025

(54) COMBINATION OF ANTIBODY-DRUG CONJUGATE AND ATR INHIBITOR

(71) Applicants: AstraZeneca UK Limited, Cambridge (GB); DAIICHI SANKYO COMPANY , LIMITED, Tokyo (JP)

(72) Inventors: Matthew Simon SUNG, Wilmington, DE (US); Alan Yin Kai LAU, Cambridge (GB); Yann WALLEZ, Cambridge (GB); Jerome Thomas METTETAL, II, Wilmington (DE); Theresa Angela PROIA, Wilmington, DE (US); Suzanne Jane RANDLE, Cambridge (GB); Mark John ANDERTON, Cambridge (GB)

(73) Assignees: AstraZeneca UK Limited, Cambridge (GB); DAIICHI SANKYO COMPANY , LIMITED, Tokyo (JP)

(21) Appl. No.: 18/724,710

(22) PCT Filed: Dec. 27, 2022
(86) PCT No.: PCT/IB2022/062798

§ 371 Date: Jun. 27, 2024

Related U.S. Application Data
(60) Provisional application No. 63/294,368, filed on Dec. 28, 2021.

(51) Int. Cl. A61K 47/68; A61K 31/5377; A61P 35/00

(52) U.S. Cl. CPC A61K 47/6851; A61K 31/5377; A61K 47/68037; A61K 47/6889; A61P 35/00

(57) ABSTRACT

A pharmaceutical product for administration of an anti TROP2 antibody-drug conjugate in combination with an ATR inhibitor is provided. The anti-TROP2 antibody-drug conjugate is an antibody-drug conjugate in which a drug linker represented by the following formula (wherein A represents the connecting position to an anti-TROP2 antibody) is conjugated to an anti-TROP2 antibody via a thioether bond. Also provided is a therapeutic use and method wherein the anti-TROP2 antibody-drug conjugate and the ATR inhibitor are administered in combination to a subject: Formula (I).

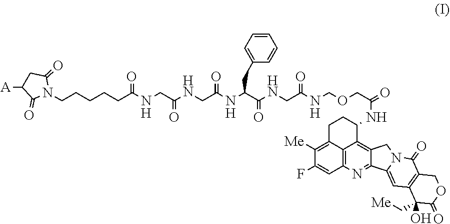

### TECHNICAL FIELD

The present disclosure relates to a pharmaceutical product for administration of a specific antibody-drug conjugate, having an antitumor drug conjugated to an anti-TROP2 antibody via a linker structure, in combination with an ATR inhibitor, and to a therapeutic use and method wherein the specific antibody-drug conjugate and the ATR inhibitor are administered in combination to a subject.

### BACKGROUND

ATR (ataxia telangiectasia and rad3-related kinase) is a serine/threonine protein kinase and member of the phosphatidylinositol 3-kinase related kinase (PIKK) family. During normal DNA replication, ATR is recruited at stalled replication forks, which can progress to double strand breaks if left unrepaired. ATR is recruited to single strand DNA coated with Replication Protein A (RPA) following single strand DNA damage or the resection of double strand breaks during DNA replication. Recruitment and activation of ATR leads to cell cycle arrest in S-phase while the DNA is repaired and the stalled replication fork resolved, or nuclear fragmentation and entry into programmed cell death (apoptosis).

As a result, ATR inhibitors are expected to cause growth inhibition in tumor cells dependent upon ATR for DNA repair e.g. ATM-deficient tumors. In addition to such monotherapy activity, ATR inhibitors are also predicted to potentiate the activity of DNA damage-inducing therapies (through inhibition of ATR-dependent DNA repair processes) when used in combination. Examples of ATR inhibitors are disclosed, for example, in WO2011/154737.

Inactivation of Schlafen 11 (SLFN11) in cancer cells has also been shown to result in resistance to anticancer agents that cause DNA damage and replication stress. Thus, SLFN11 may serve as a determinant of sensitivity to different classes of DNA-damaging agents including but not restricted to topoisomerase I inhibitors. See Zoppoli et al., PNAS 2012; 109:15030-35; Murai et al., Oncotarget 2016; 7:76534-50; Murai et al., Mol. Cell 2018; 69:371-84.

Antibody-drug conjugates (ADCs), which are composed of a cytotoxic drug conjugated to an antibody, can deliver the drug selectively to cancer cells and can thus be expected to cause accumulation of the drug within cancer cells and to kill the cancer cells (Ducry, L., et al., Bioconjugate Chem. (2010) 21, 5-13; Alley, S. C., et al., Current Opinion in Chemical Biology (2010) 14, 529-537; Damle N. K. Expert Opin. Biol. Ther. (2004) 4, 1445-1452; Senter P. D., et al., Nature Biotechnology (2012) 30, 631-637; Burris H A., et al., J. Clin. Oncol. (2011) 29(4): 398-405).

One such antibody-drug conjugate is datopotamab deruxtecan, which is composed of a TROP2-targeting antibody and a derivative of exatecan. In particular, WO 2015/098099 and WO 2020/240467 provide detailed descriptions of exemplary TROP2-targeting antibody-drug conjugates, including datopotamab deruxtecan (DS-1062). Datopotamab deruxtecan has shown clinical efficacy in multiple tumor types, including lung cancer and breast cancer. However, there is still a need to identify combination partners for anti-TROP2 antibody-drug conjugates, such as datopotamab deruxtecan, to enhance their therapeutic potential.

Despite the therapeutic potential of anti-TROP2 antibody-drug conjugates such as datopotamab deruxtecan (DS-1062) and of ATR inhibitors, a need remains for improved therapeutic compositions and methods that can enhance efficacy of existing cancer treating agents, increase durability of therapeutic response, improve tolerance to patients, reduce dose-dependent toxicity, and/or provide an alternative treatment of cancers exhibiting resistance or refractoriness to a previous cancer treatment, for example a previous treatment with a PARP inhibitor such as olaparib, rucaparib, niraparib, talazoparib or veliparib.

### SUMMARY OF DISCLOSURE

The antibody-drug conjugate used in the present disclosure (an anti-TROP2 antibody-drug conjugate that includes a derivative of the topoisomerase I inhibitor exatecan, as a component) has been confirmed to exhibit an excellent antitumor effect in the treatment of certain cancers such as breast cancer and lung cancer, when administered singly. However, it is desired to provide a medicine and treatment which can obtain a superior antitumor effect in the treatment of cancers, such as enhanced efficacy, increased durability of therapeutic response and/or reduced dose-dependent toxicity. By inhibiting the DNA damage response to replication stress and double strand breaks introduced by the antibody-drug conjugate of the present disclosure, an ATR inhibitor may further enhance antitumor efficacy when administered in combination with the antibody-drug conjugate.

The present disclosure provides a pharmaceutical product which can exhibit an excellent antitumor effect in the treatment of cancers, through administration of an anti-TROP2 antibody-drug conjugate in combination with an ATR inhibitor. The present disclosure also provides a therapeutic use and method wherein the anti-TROP2 antibody-drug conjugate and ATR inhibitor are administered in combination to a subject.

Specifically, the present disclosure relates to the following [1] to:
[1] a pharmaceutical product comprising an anti-TROP2 antibody-drug conjugate and an ATR inhibitor for administration in combination, wherein the anti-TROP2 antibody-drug conjugate is an antibody-drug conjugate in which a drug-linker represented by the following formula:

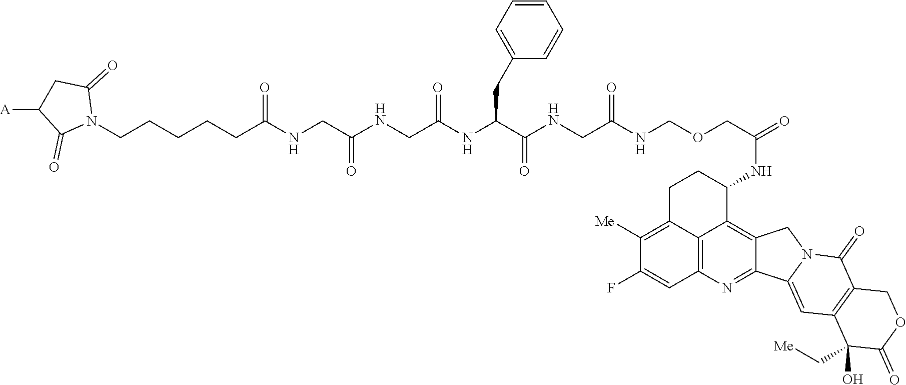

wherein A represents the connecting position to an antibody, is conjugated to an anti-TROP2 antibody via a thioether bond;
[2] the pharmaceutical product according to [1], wherein the ATR inhibitor is a compound represented by the following formula (I):

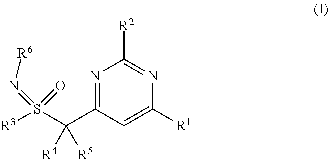

wherein:
   R¹ is selected from morpholin-4-yl and 3-methylmorpholin-4-yl;
   R² is

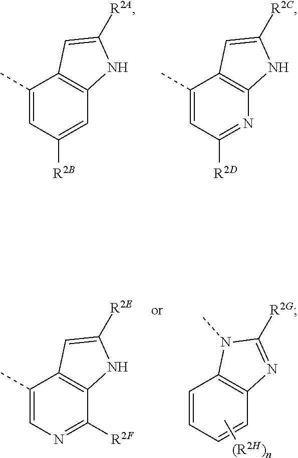

n is 0 or 1;
R^{2A}, R^{2C}, R^{2E} and R^{2F} each independently are hydrogen or methyl;
R^{2B} and R^{2D} each independently are hydrogen or methyl;
R^{2G} is selected from —NHR⁷ and —NHCOR⁸;
R^{2E} is fluoro;
R³ is methyl;
R⁴ and R⁵ are each independently hydrogen or methyl, or R⁴ and R⁵ together with the atom to which they are attached form Ring A;
Ring A is a C₃₋₆cycloalkyl or a saturated 4-6 membered heterocyclic ring containing one heteroatom selected from O and N;
R⁶ is hydrogen;
R⁷ is hydrogen or methyl;
R⁸ is methyl,
or a pharmaceutically acceptable salt thereof;
[3] the pharmaceutical product according to [2] wherein, in formula (I), R⁴ and R⁵ together with the atom to which they are attached form Ring A, and Ring A is a C₃₋₆cycloalkyl or a saturated 4-6 heterocyclic ring containing one heteroatom selected from O and N;
[4] the pharmaceutical product according to [2] or [3] wherein, in formula (I), Ring A is a cyclopropyl, tetrahydropyranyl or piperidinyl ring;
[5] the pharmaceutical product according to any one of [2] to [4] wherein, in formula (I), R^{2A} is hydrogen; R^{2B} is hydrogen; R^{2C} is hydrogen; R^{2D} is hydrogen; R^{2E} is hydrogen; and R^{2F} is hydrogen;
[6] the pharmaceutical product according to any one of [2] to [5] wherein, in formula (I), R¹ is 3-methylmorpholin-4-yl;
[7] the pharmaceutical product according to any one of [2] to [6] wherein the compound of formula (I) is a compound of formula (Ia):

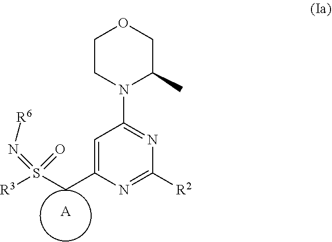

or a pharmaceutically acceptable salt thereof;
[8] the pharmaceutical product according to [7] wherein, in formula (Ia):
   Ring A is cyclopropyl ring;
   R² is

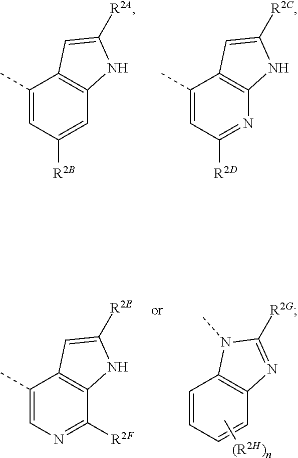

n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is —NHR⁷;
R^{2H} is fluoro;
R³ is a methyl group;
R⁶ is hydrogen; and
R⁷ is hydrogen or methyl;
[9] the pharmaceutical product according to [2], wherein the ATR inhibitor is AZD6738, also known as ceralasertib or AZ13386215, represented by the following formula:

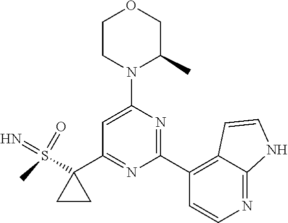 [SMILES: C[C@@H]1COCCN1c1cc(C2([S@](C)(=N)=O)CC2)nc(-c2ccnc3[nH]ccc23)n1]

or a pharmaceutically acceptable salt thereof;
[10] the pharmaceutical product according to any one of [1] to [9], wherein the anti-TROP2 antibody is an antibody comprising a heavy chain comprising CDRH1 consisting of an amino acid sequence represented by SEQ ID NO: 3 [=amino acid residues 50 to 54 of SEQ ID NO: 1], CDRH2 consisting of an amino acid sequence represented by SEQ ID NO: 4 [=amino acid residues 69 to 85 of SEQ ID NO: 1] and CDRH3 consisting of an amino acid sequence represented by SEQ ID NO: 5 [=amino acid residues 118 to 129 of SEQ ID NO: 1], and a light chain comprising CDRL1 consisting of an amino acid sequence represented by SEQ ID NO: 6 [=amino acid residues 44 to 54 of SEQ ID NO: 2], CDRL2 consisting of an amino acid sequence represented by SEQ ID NO: 7 [=amino acid residues 70 to 76 of SEQ ID NO: 2] and CDRL3 consisting of an amino acid sequence represented by SEQ ID NO: 8 [=amino acid residues 109 to 117 of SEQ ID NO: 2];
[11] the pharmaceutical product according to [10], wherein the anti-TROP2 antibody is an antibody comprising a heavy chain comprising a heavy chain variable region consisting of an amino acid sequence represented by SEQ ID NO: 9 [=amino acid residues 20 to 140 of SEQ ID NO: 1] and a light chain comprising a light chain variable region consisting of an amino acid sequence represented by SEQ ID NO: 10 [=amino acid residues 21 to 129 of SEQ ID NO: 2];
[12] the pharmaceutical product according to [11], wherein the anti-TROP2 antibody is an antibody comprising a heavy chain consisting of an amino acid sequence consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2];
[13] the pharmaceutical product according to [11], wherein the anti-TROP2 antibody is an antibody comprising a heavy chain consisting of an amino acid sequence represented by SEQ ID NO: 11 [=amino acid residues 20 to 469 of SEQ ID NO: 1] and a light chain consisting of an amino acid sequence represented by SEQ ID NO: 13 [=amino acid residues 21 to 234 of SEQ ID NO: 2];
[14] the pharmaceutical product according to any one of [1] to [13], wherein the average number of units of the drug-linker conjugated per antibody molecule in the antibody-drug conjugate is in the range of from 2 to 8;
[14] the pharmaceutical product according to [14], wherein the average number of units of the drug-linker conjugated per antibody molecule in the antibody-drug conjugate is in the range of from 3.5 to 4.5;
[15] the pharmaceutical product according to [15], wherein the anti-TROP2 antibody-drug conjugate is datopotamab deruxtecan (DS-1062);
[17] the pharmaceutical product according to any one of [1] to [16] to wherein the product is a combined preparation comprising the anti-TROP2 antibody-drug conjugate and the ATR inhibitor, for separate simultaneous administration;
[18] the pharmaceutical product according to any one of [1] to [16] to wherein the product is a combined preparation comprising the anti-TROP2 antibody-drug conjugate and the ATR inhibitor, for sequential administration;
[19] the pharmaceutical product according to any one of [1] to [18] to wherein the anti-TROP2 antibody-drug conjugate is to be administered at a dose of 6 mg/kg body weight;
[20] the pharmaceutical product according to wherein the dose of the anti-TROP2 antibody-drug conjugate is to be administered once every three weeks;
[21] the pharmaceutical product according to any one of [1] to [20] to wherein the ATR inhibitor is to be administered daily for the first week, second week, and/or third week of a three week cycle;
[22] the pharmaceutical product according to any one of [1] to [20] to wherein the ATR inhibitor is to be administered on days 3 to 17 of a three week cycle;
[23] the pharmaceutical product according to any one of [1] to [22], wherein the product is for treating cancer;
[24] the pharmaceutical product according to [23], wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma;
[25] the pharmaceutical product according to [24], wherein the cancer is breast cancer;
[26] the pharmaceutical product according to [25], wherein the breast cancer is triple negative breast cancer;
[27] the pharmaceutical product according to [25], wherein the breast cancer is hormone receptor (HR)-positive, HER2-negative breast cancer;
[28] the pharmaceutical product according to [24], wherein the cancer is lung cancer;
[29] the pharmaceutical product according to [28], wherein the lung cancer is non-small cell lung cancer;
[30] the pharmaceutical product according to [29], wherein the non-small cell lung cancer is non-small cell lung cancer with actionable genomic alterations;
[31] the pharmaceutical product according to [29], wherein the non-small cell lung cancer is non-small cell lung cancer lung cancer without actionable genomic alterations;
[32] the pharmaceutical product according to [24], wherein the cancer is colorectal cancer;
[33] the pharmaceutical product according to [24], wherein the cancer is gastric cancer;
[34] the pharmaceutical product according to [24], wherein the cancer is pancreatic cancer;
[35] the pharmaceutical product according to [24], wherein the cancer is ovarian cancer;
[36] the pharmaceutical product according to [24], wherein the cancer is prostate cancer;
[37] the pharmaceutical product according to [24], wherein the cancer is kidney cancer;
[38] the pharmaceutical product according to any one of [24] to [37], wherein the cancer is deficient in Homologous Recombination (HR) dependent DNA DSB repair activity;
[39] the pharmaceutical product according to any one of [24] to [37], wherein the cancer is not deficient in Homologous Recombination (HR) dependent DNA DSB repair activity;
[40] the pharmaceutical product according to any one of [24] to [37], wherein the cancer is exhibits resistance or refractoriness to a previous treatment with a PARP inhibitor;
[41] the pharmaceutical product according to [40], wherein the previous treatment is with a PARP inhibitor selected from olaparib, rucaparib, niraparib, talazoparib and veliparib;
[42] the pharmaceutical product according to any one of [24] to [38], wherein cancer cells of the cancer are SLFN11-deficient;
[43] the pharmaceutical product according to [42], wherein SLFN11 expression is lower in the cancer cells of a patient relative to the patient's SLFN11-expressing non-cancer cells;
[44] a pharmaceutical product as defined in any one of [1] to [22], for use in treating cancer;
[45] the pharmaceutical product for the use according to [44], wherein the cancer is as defined in any one of [24] to [43];
[46] use of an anti-TROP2 antibody-drug conjugate in the manufacture of a medicament for use in combination with an ATR inhibitor, wherein the anti-TROP2 antibody-drug conjugate and the ATR inhibitor are as defined in any one of [1] to [16], for treating cancer;
[47] the use according to wherein the medicament is for use in combination with the ATR inhibitor by sequential administration;
[48] the use according to wherein the medicament is for use in combination with the ATR inhibitor by separate simultaneous administration;
[49] use of an ATR inhibitor in the manufacture of a medicament for use in combination with an anti-TROP2 antibody-drug conjugate, wherein the anti-TROP2 antibody-drug conjugate and the ATR inhibitor are as defined in any one of [1] to [16], for treating cancer;
[50] the use according to wherein the medicament is for use in combination with the anti-TROP2 antibody-drug conjugate by sequential administration;
[51] the use according to wherein the medicament is for use in combination with the anti-TROP2 antibody-drug conjugate by separate simultaneous administration;
[52] the use according to any one of to [51], wherein the cancer is as defined in any one of to [43];
[53] an anti-TROP2 antibody-drug conjugate for use, in combination with an ATR inhibitor, in the treatment of cancer, wherein the anti-TROP2 antibody-drug conjugate and the ATR inhibitor are as defined in any one of [1] to [16];
[54] the anti-TROP2 antibody-drug conjugate for the use according to [53], wherein the cancer is as defined in any one of to [43];
[55] the anti-TROP2 antibody-drug conjugate for the use according to or [54], wherein the use comprises administration of the anti-TROP2 antibody-drug conjugate and the ATR inhibitor sequentially;
[56] the anti-TROP2 antibody-drug conjugate for the use according to or [54], wherein the use comprises administration of the anti-TROP2 antibody-drug conjugate and the ATR inhibitor separately and simultaneously;
[57] an anti-TROP2 antibody-drug conjugate for use in the treatment of cancer in a subject, wherein said treatment comprises the sequential or separate simultaneous administration of i) said anti-TROP2 antibody-drug conjugate, and ii) an ATR inhibitor to said subject, wherein said anti-TROP2 antibody-drug conjugate and said ATR inhibitor are as defined in any one of [1] to [16];
[58] the anti-TROP2 antibody-drug conjugate for the use according to any one of to wherein the anti-TROP2 antibody-drug conjugate is to be administered at a dose of 6 mg/kg body weight;
[59] the anti-TROP2 antibody-drug conjugate for the use according to wherein the dose of the anti-TROP2 antibody-drug conjugate is to be administered once every three weeks;
[60] the anti-TROP2 antibody-drug conjugate for the use according to any one of to wherein the ATR inhibitor is to be administered daily for the first week, second week, and/or third week of a three week cycle;
[61] the anti-TROP2 antibody-drug conjugate for the use according to any one of to wherein the ATR inhibitor is to be administered on days 3 to 17 of a three week cycle;
[62] an ATR inhibitor for use, in combination with an anti-TROP2 antibody-drug conjugate, in the treatment of cancer, wherein the anti-TROP2 antibody-drug conjugate and the ATR inhibitor are as defined in any one of [1] to [16];
[63] the ATR inhibitor for the use according to [62], wherein the cancer is as defined in any one of to [43];
[64] the ATR inhibitor for the use according to [62] or [63], wherein the use comprises administration of the anti-TROP2 antibody-drug conjugate and the ATR inhibitor sequentially;
[65] the ATR inhibitor for the use according to [62] or [63], wherein the use comprises administration of the anti-TROP2 antibody-drug conjugate and the ATR inhibitor separately and simultaneously;
[66] an ATR inhibitor for use in the treatment of cancer in a subject, wherein said treatment comprises the, sequential or separate simultaneous administration of i) said ATR inhibitor, and ii) an anti-TROP2 antibody-drug conjugate to said subject, wherein said ATR inhibitor and said anti-TROP2 antibody-drug conjugate are as defined in any one of [1] to [16];
[67] the ATR inhibitor for the use according to any one of [62] to [66] wherein the anti-TROP2 antibody-drug conjugate is to be administered at a dose of 6 mg/kg body weight;
[68] the ATR inhibitor for the use according to [67] wherein the dose of the anti-TROP2 antibody-drug conjugate is to be administered once every three weeks.
[69] the ATR inhibitor for the use according to any one of [62] to [68] wherein the ATR inhibitor is to be administered daily for the first week, second week, and/or third week of a three week cycle;
[70] the ATR inhibitor for the use according to any one of [62] to [68] wherein the ATR inhibitor is to be administered on days 3 to 17 of a three week cycle;
[71] a method of treating cancer comprising administering an anti-TROP2 antibody-drug conjugate and an ATR inhibitor as defined in any one of [1] to [16] in combination to a subject in need thereof;
[72] the method according to [71], wherein the cancer is as defined in any one of [24] to [43];
[73] the method according to [71] or [72], wherein the method comprises administering the anti-TROP2 antibody-drug conjugate and the ATR inhibitor sequentially; and
[74] the method according to [71] or [72], wherein the method comprises administering the anti-TROP2 antibody-drug conjugate and the ATR inhibitor separately and simultaneously.

### Advantageous Effects of Disclosure

The present disclosure provides a pharmaceutical product wherein an anti-TROP2 antibody-drug conjugate, having an antitumor drug conjugated to an anti-TROP2 antibody via a linker structure, and an ATR inhibitor are administered in combination, and a therapeutic use and method wherein the anti-TROP2 antibody-drug conjugate and the ATR inhibitor are administered in combination to a subject. Thus, the present disclosure can provide a medicine and treatment which can obtain a superior antitumor effect in the treatment of cancers.

### BRIEF DESCRIPTION OF DRAWINGS

FIG. 1 is a diagram showing the amino acid sequence of a heavy chain of an anti-TROP2 antibody (SEQ ID NO: 1).

FIG. 2 is a diagram showing the amino acid sequence of a light chain of an anti-TROP2 antibody (SEQ ID NO: 2).

FIG. 3 is a diagram showing the amino acid sequence of a heavy chain CDRH1 (SEQ ID NO: 3 [=amino acid residues 50 to 54 of SEQ ID NO: 1]).

FIG. 4 is a diagram showing the amino acid sequence of a heavy chain CDRH2 (SEQ ID NO: 4 [=amino acid residues 69 to 85 of SEQ ID NO: 1]).

FIG. 5 is a diagram showing the amino acid sequence of a heavy chain CDRH3 (SEQ ID NO: 5 [=amino acid residues 118 to 129 of SEQ ID NO: 1]).

FIG. 6 is a diagram showing the amino acid sequence of a light chain CDRL1 (SEQ ID NO: 6 [=amino acid residues 44 to 54 of SEQ ID NO: 2]).

FIG. 7 is a diagram showing the amino acid sequence of a light chain CDRL2 (SEQ ID NO: 7 [=amino acid residues 70 to 76 of SEQ ID NO: 2]).

FIG. 8 is a diagram showing the amino acid sequence of a light chain CDRL3 (SEQ ID NO: 8 [=amino acid residues 109 to 117 of SEQ ID NO: 2]).

FIG. 9 is a diagram showing the amino acid sequence of a heavy chain variable region (SEQ ID NO: 9 [=amino acid residues 20 to 140 of SEQ ID NO: 1]).

FIG. 10 is a diagram showing the amino acid sequence of a light chain variable region (SEQ ID NO: 10 [=amino acid residues 21 to 129 of SEQ ID NO: 2]).

FIG. 11 is a diagram showing the amino acid sequence of a heavy chain (SEQ ID NO: 11 [=amino acid residues 20 to 469 of SEQ ID NO: 1]). [FIGS. 12A and 12B]FIGS. 12A and 12B are diagrams showing combination matrices obtained with high-throughput screens combining DS-1062 with AZD6738 (ATR inhibitor) in TROP2-expressing lung cancer cell lines. [FIGS. 13A and 13B]FIGS. 13A and 13B are diagrams showing combination matrices obtained with high-throughput screens combining DS-1062 with AZD6738 in TROP2-expressing breast cancer cell lines. FIG. 14 is a diagram showing combination Emax and Loewe synergy scores in cell lines treated with DS-1062 combined with AZD6738. FIG. 15 is a graph showing tumor volumes for in vivo treatments with DS-1062 or AZD6738 alone or with DS-1062 in combination with AZD6738. The dotted line represents the end of the AZD6738 dosing period. [FIGS. 16A and 16B]FIG. 16A is a graph showing tumor volumes for in vivo treatments with DS-1062 or AZD6738 alone or with DS-1062 in combination with AZD6738. The dotted line represents the end of the AZD6738 dosing period. FIG. 16B is a graph of percentage of mice achieving a complete response in each treatment group from this study.

FIGS. 17A and 17B are diagrams showing combination matrices obtained with high-throughput screens combining DS-1062 with AZD6738 in primary CD34+ hematopoietic stem and progenitors cells differentiated along the erythroid lineage.

FIG. 18 is a graph showing tumor volumes for in vivo treatments with DS-1062 or AZD6738 alone or with DS-1062 in combination with AZD6738.

FIG. 19 is a graph showing tumor volumes for in vivo treatments with DS-1062 or AZD6738 alone or with DS-1062 in combination with AZD6738. The dotted line represents the end of the dosing periods.

FIG. 20 is a graph showing tumor volumes for in vivo treatments with DS-1062 or AZD6738 alone or with DS-1062 in combination with AZD6738. The dotted line represents the end of the dosing period.

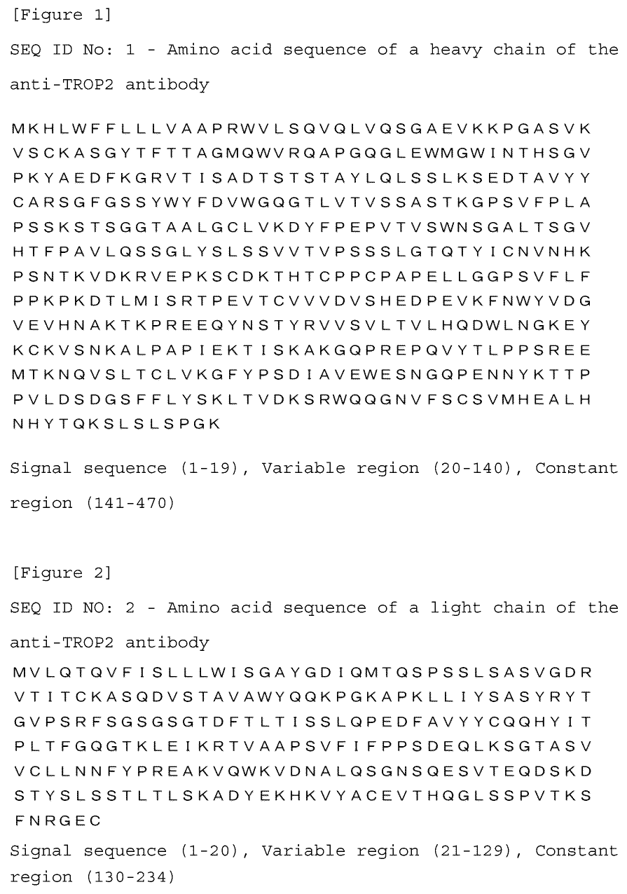

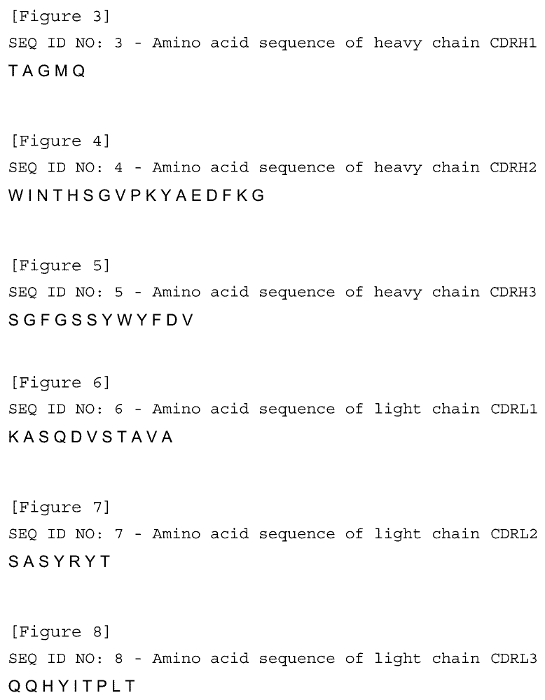

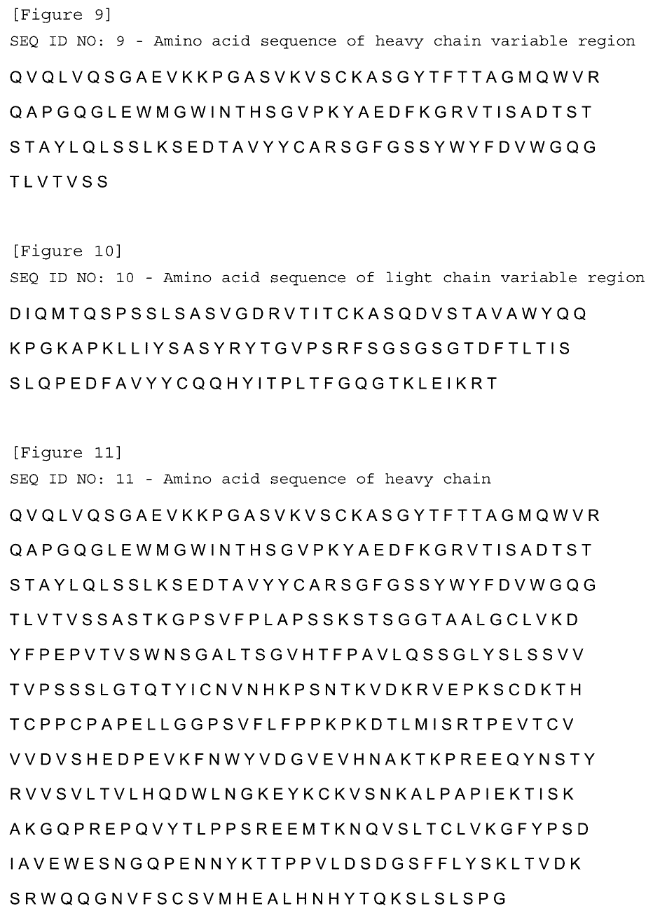

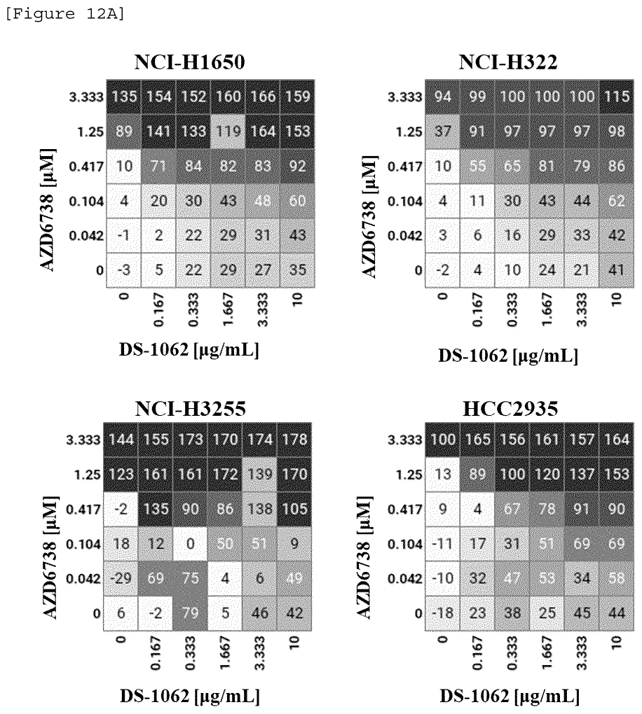

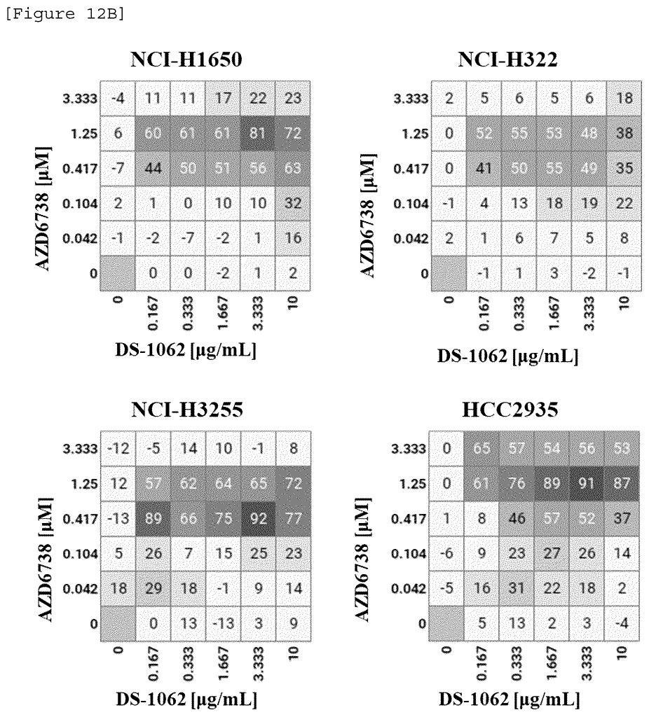

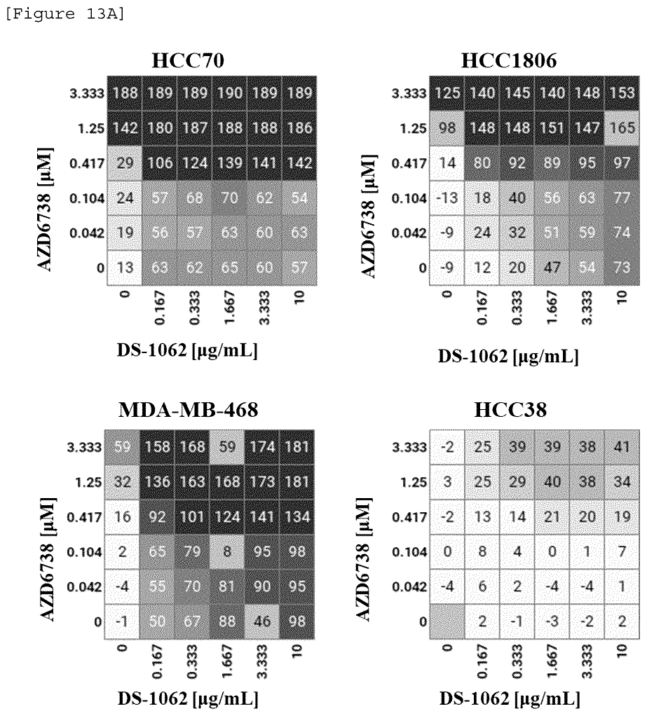

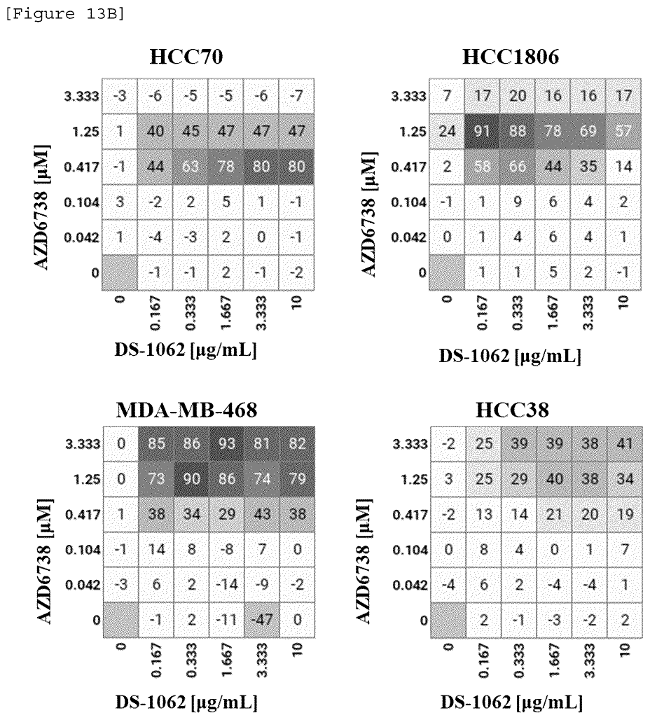

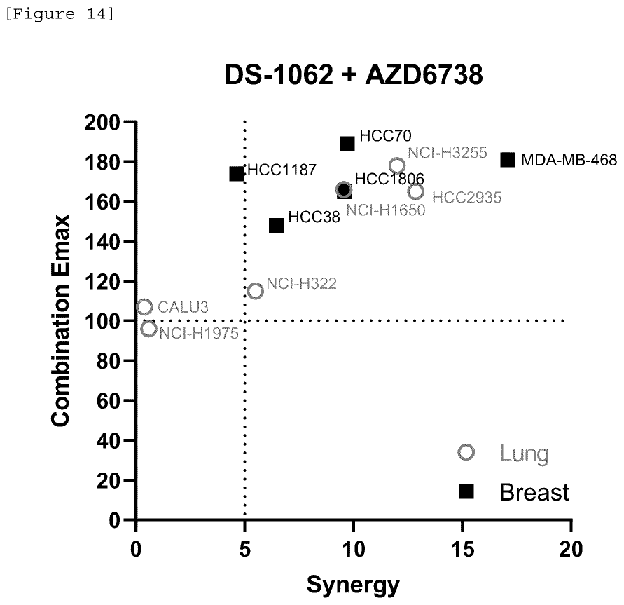

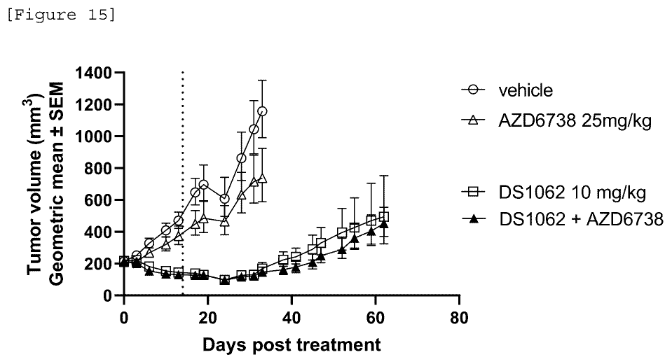

In order that the present disclosure can be more readily understood, certain terms are first defined. Additional definitions are set forth throughout the detailed description.

Before describing the present disclosure in detail, it is to be understood that this disclosure is not limited to specific compositions or method steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range.

It is understood that wherever aspects are described herein with the language “comprising”, otherwise analogous aspects described in terms of “consisting of” and/or “consisting essentially of” are also provided.

The terms “inhibit”, “block”, and “suppress” are used interchangeably herein and refer to any statistically significant decrease in biological activity, including full blocking of the activity. For example, “inhibition” can refer to a decrease of about 10%, 20%, 30%, 40%, 50%, 60%, 70%, 80%, 90% or 100% in biological activity. Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

The term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

The term “pharmaceutical product” refers to a preparation which is in such form as to permit the biological activity of the active ingredients, either as a composition containing all the active ingredients (for simultaneous administration), or as a combination of separate compositions (a combined preparation) each containing at least one but not all of the active ingredients (for administration sequentially or simultaneously), and which contains no additional components which are unacceptably toxic to a subject to which the product would be administered. Such product can be sterile. By “simultaneous administration” is meant that the active ingredients are administered at the same time. By “sequential administration” is meant that the active ingredients are administered one after the other, in either order, at a time interval between the individual administrations. The time interval can be, for example, less than 24 hours, preferably less than 6 hours, more preferably less than 2 hours.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain aspects, a subject is successfully “treated” for cancer according to the methods of the present disclosure if the patient shows, e.g., total, partial, or transient remission of a certain type of cancer.

The terms “cancer”, “tumor”, “cancerous”, and “malignant” refer to or describe the physiological condition in mammals that is typically characterized by unregulated cell growth. Examples of cancers include but are not limited to, breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma. Cancers include hematological malignancies such as acute myeloid leukemia, multiple myeloma, chronic lymphocytic leukemia, diffuse large B cell lymphoma, Burkitt's lymphoma, follicular lymphoma and solid tumors such as breast cancer, lung cancer, neuroblastoma and colon cancer.

The term “cytotoxic agent” as used herein is defined broadly and refers to a substance that inhibits or prevents the function of cells and/or causes destruction of cells (cell death), and/or exerts anti-neoplastic/anti-proliferative effects. For example, a cytotoxic agent prevents directly or indirectly the development, maturation, or spread of neoplastic tumor cells. The term includes also such agents that cause a cytostatic effect only and not a mere cytotoxic effect. The term includes chemotherapeutic agents as specified below, as well as other TROP2 antagonists, anti-angiogenic agents, tyrosine kinase inhibitors, protein kinase A inhibitors, members of the cytokine family, radioactive isotopes, and toxins such as enzymatically active toxins of bacterial, fungal, plant or animal origin. The term “chemotherapeutic agent” is a subset of the term “cytotoxic agent” comprising natural or synthetic chemical compounds.

In accordance with the methods or uses of the present disclosure, compounds of the present disclosure may be administered to a patient to promote a positive therapeutic response with respect to cancer. The term “positive therapeutic response” with respect to cancer treatment refers to an improvement in the symptoms associated with the disease. For example, an improvement in the disease can be characterized as a complete response. The term “complete response” refers to an absence of clinically detectable disease with normalization of any previous test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of compounds of the present disclosure. In specific aspects, such terms refer to one, two or three or more results following the administration of compounds of the instant disclosure:
(1) a stabilization, reduction or elimination of the cancer cell population;
(2) a stabilization or reduction in cancer growth; (3) an impairment in the formation of cancer;
(4) eradication, removal, or control of primary, regional and/or metastatic cancer;
(5) a reduction in mortality;
(6) an increase in disease-free, relapse-free, progression-free, and/or overall survival, duration, or rate;
(7) an increase in the response rate, the durability of response, or number of patients who respond or are in remission;
(8) a decrease in hospitalization rate,
(9) a decrease in hospitalization lengths,
(10) the size of the cancer is maintained and does not increase or increases by less than 10%, preferably less than 5%, preferably less than 4%, preferably less than 2%, and
(11) an increase in the number of patients in remission.
(12) a decrease in the number of adjuvant therapies (e.g., chemotherapy or hormonal therapy) that would otherwise be required to treat the cancer.

Clinical response can be assessed using screening techniques such as PET, magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy can experience the beneficial effect of an improvement in the symptoms associated with the disease.

As used herein, the term “the expression level of SLFN11 is” some amount, e.g. 0%, means that the stated amount of cancer cells in the patient's cancer tissue express SLFN11. Similarly, as used herein, the term “the expression level of SLFN11 is <” some amount, e.g. 10%, means that less than the stated amount of cancer cells in the patient's cancer tissue express SLFN11. The expression level of SLFN11 may be, for example, <25%, <20%, <15%, <10%, <9%, <8%, <7%, <6%, <5%, <4%, <3%, <2%, <1% or 0%.

As used herein, the term “SLFN11-deficient” refers to an expression level of SLFN11 in the relevant patient, animal, tissue, cell, etc. that is inadequate to exhibit the normal phenotype associated with the gene, or for the protein to exhibit its physiological function. In the context of preclinical models, cells or animals in which the SLFN11 gene is knocked out (KO) are examples of “SLFN11-deficient”.

In this specification the generic term “C_{p-q}alkyl” includes both straight-chain and branched-chain alkyl groups. However references to individual alkyl groups such as “propyl” are specific for the straight chain version only (i.e. n-propyl and isopropyl) and references to individual branched-chain alkyl groups such as “tert-butyl” are specific for the branched chain version only.

The prefix C_{p-q} in C_{p-q}alkyl and other terms (where p and q are integers) indicates the range of carbon atoms that are present in the group, for example C₁₋₄alkyl includes C₁alkyl (methyl), C₂alkyl (ethyl), C₃alkyl (propyl as n-propyl and isopropyl) and C₄alkyl (n-butyl, sec-butyl, isobutyl and tert-butyl).

The term C_{p-q}alkoxy comprises —O—C_{p-q}alkyl groups.

The term C_{p-q}alkanoyl comprises —C(O)alkyl groups.

The term halo includes fluoro, chloro, bromo and iodo.

“Carbocyclyl” is a saturated, unsaturated or partially saturated monocyclic ring system containing from 3 to 6 ring atoms, wherein a ring CH₂ group may be replaced with a C═O group. “Carbocyclyl” includes “aryl”, “C_{p-q}cycloalkyl” and “C_{p-q}cycloalkenyl”.

“Aryl” is an aromatic monocyclic carbocyclyl ring system.

“C_{p-q}cycloalkenyl” is an unsaturated or partially saturated monocyclic carbocyclyl ring system containing at least 1 C═C bond and wherein a ring CH₂ group may be replaced with a C═O group.

“C_{p-q}cycloalkyl” is a saturated monocyclic carbocyclyl ring system and wherein a ring CH₂ group may be replaced with a C═O group.

“Heterocyclyl” is a saturated, unsaturated or partially saturated monocyclic ring system containing from 3 to 6 ring atoms of which 1, 2 or 3 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group. “Heterocyclyl” includes “heteroaryl”, “cycloheteroalkyl” and “cycloheteroalkenyl”.

“Heteroaryl” is an aromatic monocyclic heterocyclyl, particularly having 5 or 6 ring atoms, of which 1, 2 or 3 ring atoms are chosen from nitrogen, sulfur or oxygen where a ring nitrogen or sulfur may be oxidised.

“Cycloheteroalkenyl” is an unsaturated or partially saturated monocyclic heterocyclyl ring system, particularly having 5 or 6 ring atoms, of which 1, 2 or 3 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

“Cycloheteroalkyl” is a saturated monocyclic heterocyclic ring system, particularly having 5 or 6 ring atoms, of which 1, 2 or 3 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

This specification may make use of composite terms to describe groups comprising more than one functionality. Unless otherwise described herein, such terms are to be interpreted as is understood in the art. For example carbocyclylC_{p-q}alkyl comprises C_{p-q}alkyl substituted by carbocyclyl, heterocyclylC_{p-q}alkyl comprises C_{p-q}alkyl substituted by heterocyclyl, and bis(C_{p-q}alkyl)amino comprises amino substituted by 2 C_{p-q}alkyl groups which may be the same or different. HaloC_{p-q}alkyl is a C_{p-q}alkyl group that is substituted by 1 or more halo substituents and particularly 1, 2 or 3 halo substituents. Similarly, other generic terms containing halo such as haloC_{p-q}alkoxy may contain 1 or more halo substituents and particularly 1, 2 or 3 halo substituents.

HydroxyC_{p-q}alkyl is a C_{p-q}alkyl group that is substituted by 1 or more hydroxyl substituents and particularly by 1, 2 or 3 hydroxy substituents. Similarly other generic terms containing hydroxy such as hydroxyC_{p-q}alkoxy may contain 1 or more and particularly 1, 2 or 3 hydroxy substituents.

C_{p-q}alkoxyC_{p-q}alkyl is a C_{p-q}alkyl group that is substituted by 1 or more C_{p-q}alkoxy substituents and particularly 1, 2 or 3 C_{p-q}alkoxy substituents. Similarly other generic terms containing C_{p-q}alkoxy such as C_{p-q}alkoxyC_{p-q}alkoxy may contain 1 or more C_{p-q}alkoxy substituents and particularly 1, 2 or 3 C_{p-q}alkoxy substituents.

Where optional substituents are chosen from “1 or 2”, from “1, 2, or 3” or from “1, 2, 3 or 4” groups or substituents it is to be understood that this definition includes all substituents being chosen from one of the specified groups i.e. all substitutents being the same or the substituents being chosen from two or more of the specified groups i.e. the substitutents not being the same.

Compounds of the present disclosure have been named with the aid of computer software (ACD/Name version 10.06).

Suitable values for any R group or any part or substituent for such groups include:
for C₁₋₃alkyl: methyl, ethyl, propyl and iso-propyl;
for C₁₋₆alkyl: C₁₋₃alkyl, butyl, 2-methylpropyl, tert-butyl, pentyl, 2,2-dimethylpropyl, 3-methylbutyl and hexyl;
for C₃₋₆cycloalkyl: cyclopropyl, cyclobutyl, cyclopentyl and cyclohexyl;
for C₃₋₆cycloalkylC₁₋₃alkyl: cyclopropylmethyl, cyclopropylethyl, cyclobutylmethyl, cyclopentylmethyl and cyclohexylmethyl;
for aryl: phenyl;
for arylC₁₋₃alkyl: benzyl and phenethyl;
for carbocylyl: aryl, cyclohexenyl and C₃₋₆cycloalkyl;
for halo: fluoro, chloro, bromo and iodo;
for C₁₋₃alkoxy: methoxy, ethoxy, propoxy and isopropoxy;
for C₁₋₆alkoxy: C₁₋₃alkoxy, butoxy, tert-butoxy, pentyloxy, 1-ethylpropoxy and hexyloxy;
for C₁₋₃alkanoyl: acetyl and propanoyl;
for C₁₋₆alkanoyl: acetyl, propanoyl and 2-methylpropanoyl;
for heteroaryl:pyridinyl, imidazolyl, pyrimidinyl, thienyl, pyrrolyl, pyrazolyl, thiazolyl, thiazolyl, triazolyl, oxazolyl, isoxazolyl, furanyl, pyridazinyl and pyrazinyl;
for heteroarylC₁₋₃alkyl: pyrrolylmethyl, pyrrolylethyl, imidazolylmethyl, imidazolylethyl, pyrazolylmethyl, pyrazolylethyl, furanylmethyl, furanylethyl, thienylmethyl, theinylethyl, pyridinylmethyl, pyridinylethyl, pyrazinylmethyl, pyrazinylethyl, pyrimidinylmethyl, pyrimidinylethyl, pyrimidinylpropyl, pyrimidinylbutyl, imidazolylpropyl, imidazolylbutyl, 1,3,4-triazolylpropyl and oxazolylmethyl;
for heterocyclyl: heteroaryl, pyrrolidinyl, piperidinyl, piperazinyl, azetidinyl, morpholinyl, dihydro-2H-pyranyl, tetrahydropyridine and tetrahydrofuranyl;
for saturated heterocyclyl: oxetanyl, pyrrolidinyl, piperidinyl, piperazinyl, azetidinyl, morpholinyl, tetrahydropyranyl and tetrahydrofuranyl.

It should be noted that examples given for terms used in the description are not limiting.

As used herein, the phrase “effective amount” means an amount of a compound or composition which is sufficient enough to significantly and positively modify the symptoms and/or conditions to be treated (e.g., provide a positive clinical response). The effective amount of an active ingredient for use in a pharmaceutical product will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician.

In particular, an effective amount of a compound of formula (I) for use in the treatment of cancer in combination with the antibody-drug conjugate is an amount such that the combination is sufficient to symptomatically relieve in a warm-blooded animal such as man, the symptoms of cancer, to slow the progression of cancer, or to reduce in patients with symptoms of cancer the risk of getting worse.

As used herein, the term “pharmaceutically acceptable” refers to those compounds, materials, compositions, and/or dosage forms which are, within the scope of sound medical judgement, suitable for use in contact with the tissues of human beings and animals without excessive toxicity, irritation, allergic response, or other problem or complication, commensurate with a reasonable benefit/risk ratio.

Certain compounds of formula (I) are capable of existing in stereoisomeric forms. It will be understood that the disclosure encompasses all geometric and optical isomers of the compounds of formula (I) and mixtures thereof including racemates. Tautomers and mixtures thereof also form an aspect of the present disclosure.

Solvates and mixtures thereof also form an aspect of the present disclosure. For example, a suitable solvate of a compound of formula (I) is, for example, a hydrate such as a hemi-hydrate, a mono-hydrate, a di-hydrate or a tri-hydrate or an alternative quantity thereof.

It is to be understood that, insofar as certain of the compounds of formula (I) defined above may exist in optically active or racemic forms by virtue of one or more asymmetric carbon atoms or sulphur atoms, the disclosure includes in its definition any such optically active or racemic form which possesses the above-mentioned activity. The present disclosure encompasses all such stereoisomers having activity as herein defined. It is further to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ratio 50:50. The synthesis of optically active forms may be carried out by standard techniques of organic chemistry well known in the art, for example by synthesis from optically active starting materials or by resolution of a racemic form. Racemates may be separated into individual enantiomers using known procedures (see, for example, Advanced Organic Chemistry: 3rd Edition: author J March, p 104-107). A suitable procedure involves formation of diastereomeric derivatives by reaction of the racemic material with a chiral auxiliary, followed by separation, for example by chromatography, of the diastereomers and then cleavage of the auxiliary species. Similarly, the above-mentioned activity may be evaluated using standard laboratory techniques.

It will be understood that compounds of formula (I) may encompass compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

The present disclosure may use compounds of formula (I) as herein defined as well as to salts thereof. Salts for use in pharmaceutical products will be pharmaceutically acceptable salts, but other salts may be useful in the production of the compounds of formula (I) and their pharmaceutically acceptable salts.

Pharmaceutically acceptable salts of the disclosure may, for example, include acid addition salts of compounds of formula (I) as herein defined which are sufficiently basic to form such salts. Such acid addition salts include but are not limited to fumarate, methanesulfonate, hydrochloride, hydrobromide, citrate and maleate salts and salts formed with phosphoric and sulfuric acid. In addition where compounds of formula (I) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of formula (I) may also be provided as in vivo hydrolysable esters. An in vivo hydrolysable ester of a compound of formula (I) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkcarbonyloxyC₁₋₆alkyl esters for example 1-cyclohexylcarbonyloxyethyl, (1,3-dioxolen-2-one)ylmethyl esters for example (5-methyl-1,3-dioxolen-2-one)ylmethyl, and C₁₋₆alkoxycarbonyloxyethyl esters for example 1-methoxycarbonyloxyethyl; and may be formed at any carboxy group in the compounds of this disclosure.

Suitable pharmaceutically acceptable esters for hydroxy include inorganic esters such as phosphate esters (including phosphoramidic cyclic esters) and a-acyloxyalkyl ethers and related compounds which as a result of the in vivo hydrolysis of the ester breakdown to give the parent hydroxy groups. Examples of a-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example acetyl, benzoyl, phenylacetyl, substituted benzoyl and phenylacetyl; C₁₋₁₀alkoxycarbonyl (to give alkyl carbonate esters), for example ethoxycarbonyl; di-C₁₋₄alkylcarbamoyl and N-(di-C₁₋₄alkylaminoethyl)-N—C₁₋₄alkylcarbamoyl (to give carbamates); di-C₁₋₄alkylaminoacetyl and carboxyacetyl. Examples of ring substituents on phenylacetyl and benzoyl include aminomethyl, C₁₋₄alkylaminomethyl and di-(C₁₋₄alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)OC₁₋₆alkyl-CO—, wherein R^{A} is for example, benzyloxy-C₁₋₄alkyl, or phenyl. Suitable substituents on a phenyl group in such esters include, for example, 4-Cr-4alkylpiperazino-Cr-4alkyl, piperazino-C₁₋₄alkyl and morpholino-C₁₋₄alkyl.

The compounds of the formula (I) may be also be administered in the form of a prodrug which is broken down in the human or animal body to give a compound of the formula (I). Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

### DESCRIPTION OF EMBODIMENTS

Hereinafter, preferred modes for carrying out the present disclosure are described. The embodiments described below are given merely for illustrating one example of a typical embodiment of the present disclosure and are not intended to limit the scope of the present disclosure.

### 1. Antibody-Drug Conjugate

The antibody-drug conjugate used in the present disclosure is an antibody-drug conjugate in which a drug-linker represented by the following formula:

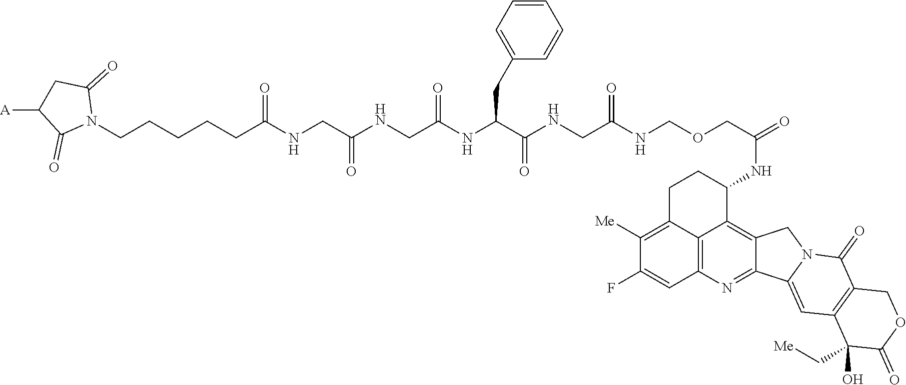

wherein A represents the connecting position to an antibody,
is conjugated to an anti-TROP2 antibody via a thioether bond.

In the present disclosure, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present disclosure includes exatecan (IUPAC name: (1S,9S)-1-amino-9-ethyl-5-fluoro-1,2,3,9,12,15-hexahydro-9-hydroxy-4-methyl-10H,13H-benzo[de]pyrano[3′,4′:6,7]indolizino[1,2-b]quinolin-10,13-dione, (also expressed as chemical name: (1S,9S)-1-amino-9-ethyl-5-fluoro-2,3-dihydro-9-hydroxy-4-methyl-1H,12H-benzo[de]pyrano[3′,4′:6,7]indolizino[1,2-b]quinolin-10,13(9H,15H)-dione)), which is a topoisomerase I inhibitor, as a component. Exatecan is a camptothecin derivative having an antitumor effect, represented by the following formula:

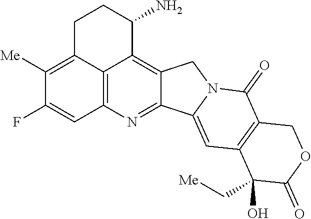 [SMILES: CC[C@@]1(O)C(=O)OCc2c1cc1n(c2=O)Cc2c-1nc1cc(F)c(C)c3c1c2[C@@H](N)CC3]

The anti-TROP2 antibody-drug conjugate used in the present disclosure can be also represented by the following formula:

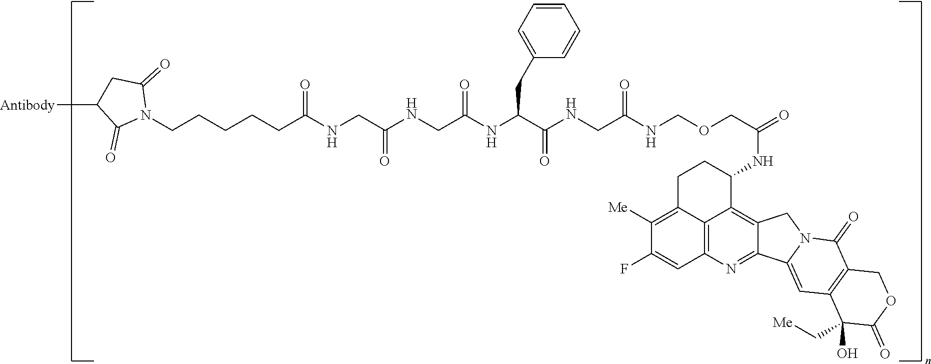

Here, the drug-linker is conjugated to an anti-TROP2 antibody (‘Antibody-’) via a thioether bond. The meaning of n is the same as that of what is called the average number of conjugated drug molecules (DAR; Drug-to-Antibody Ratio), and indicates the average number of units of the drug-linker conjugated per antibody molecule.

After migrating into cancer cells, the anti-TROP2 antibody-drug conjugate used in the present disclosure is cleaved at the linker portion to release a compound represented by the following formula:

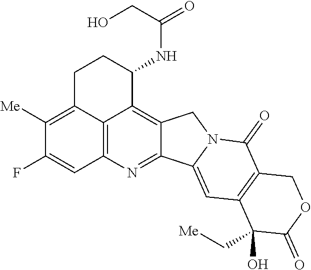 [SMILES: CC[C@@]1(O)C(=O)OCc2c1cc1n(c2=O)Cc2c-1nc1cc(F)c(C)c3c1c2[C@@H](NC(=O)CO)CC3]

### 2. Anti-TROP2 Antibody in Antibody-Drug Conjugate

The anti-TROP2 antibody in the antibody-drug conjugate used in the present invention may be derived from any species and is preferably an antibody derived from a human, a rat, a mouse, or a rabbit. In cases when the antibody is derived from species other than human species, it is preferably chimerized or humanized using a well known technique. The antibody of the present invention may be a polyclonal antibody or a monoclonal antibody and is preferably a monoclonal antibody.

The antibody in the antibody-drug conjugate used in the present invention is an antibody preferably having the characteristic of being able to target cancer cells, and is preferably an antibody possessing, for example, the property of being able to recognize a cancer cell, the property of being able to bind to a cancer cell, the property of being internalized in a cancer cell, and/or cytocidal activity against cancer cells.

The binding activity of the antibody against cancer cells can be confirmed using flow cytometry. The internalization of the antibody into tumor cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282, December 2004), or (3) a Mab-ZAP assay using an immunotoxin binding to the therapeutic antibody wherein the toxin is released upon incorporation into cells to inhibit cell growth (Bio Techniques 28: 162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining changes in the cancer cells.

Since the compound conjugated in the antibody-drug conjugate exerts an antitumor effect, it is preferred but not essential that the antibody itself should have an antitumor effect. For the purpose of specifically and selectively exerting the cytotoxic activity of the antitumor compound against cancer cells, it is important and also preferred that the antibody should have the property of being internalized to migrate into cancer cells.

The antibody in the antibody-drug conjugate used in the present invention can be obtained by a procedure known in the art. For example, the antibody of the present invention can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen can be fused with myeloma cells according to a method known in the art (for example, Kohler and Milstein, Nature (1975) 256, p. 495-497; Kennet, R. ed., Monoclonal Antibodies, p. 365-367, Plenum Press, N.Y. (1980)), to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The antibody in the antibody-drug conjugate used in the present invention is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002) 43 (7), p. 2301-2308; Carmen, S. et. al., Briefings in Functional Genomics and Proteomics (2002), 1 (2), p. 189-203; Siriwardena, D. et. al., Ophthalmology (2002) 109 (3), p. 427-431, etc.) can be exemplified.

In the antibody in the antibody-drug conjugate used in present invention, modified variants of the antibody are also included. The modified variant refers to a variant obtained by subjecting the antibody according to the present invention to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present invention, for example, an enzyme-labeled antibody, a fluorescence-labeled antibody, and an affinity-labeled antibody are also included in the meaning of the modified variant. Such a modified variant of the antibody according to the present invention is useful for improving the stability and blood retention of the antibody, reducing the antigenicity thereof, detecting or isolating an antibody or an antigen, and so on.

Further, by regulating the modification of a glycan which is linked to the antibody according to the present invention (glycosylation, defucosylation, etc.), it is possible to enhance antibody-dependent cellular cytotoxic activity. As the technique for regulating the modification of a glycan of antibodies, International Publication No. WO 99/54342, International Publication No. WO 00/61739, International Publication No. WO 02/31140, International Publication No. WO 2007/133855, International Publication No. WO 2013/120066, etc. are known. However, the technique is not limited thereto. In the antibody according to the present invention, antibodies in which the modification of a glycan is regulated are also included.

It is known that a lysine residue at the carboxyl terminus of the heavy chain of an antibody produced in a cultured mammalian cell is deleted (Journal of Chromatography A, 705: 129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360: 75-83 (2007)). However, such deletion and modification of the heavy chain sequence do not affect the antigen-binding affinity and the effector function (complement activation, antibody-dependent cellular cytotoxicity, etc.) of the antibody. Therefore, in the antibody according to the present invention, antibodies subjected to such modification and functional fragments of the antibody are also included, and deletion variants in which one or two amino acids have been deleted at the carboxyl terminus of the heavy chain, variants obtained by amidation of the deletion variants (for example, a heavy chain in which the carboxyl terminal proline residue has been amidated), and the like are also included. The type of deletion variant having a deletion at the carboxyl terminus of the heavy chain of the antibody according to the present invention is not limited to the above variants as long as the antigen-binding affinity and the effector function are conserved. The two heavy chains constituting the antibody according to the present invention may be of one type selected from the group consisting of a full-length heavy chain and the above-described deletion variant, or may be of two types in combination selected therefrom. The ratio of the amount of each deletion variant can be affected by the type of cultured mammalian cells which produce the antibody according to the present invention and the culture conditions; however, an antibody in which one amino acid residue at the carboxyl terminus has been deleted in both of the two heavy chains in the antibody according to the present invention can be preferably exemplified.

As isotypes of the antibody according to the present invention, for example, IgG (IgG1, IgG2, IgG3, IgG4) can be exemplified. Preferably, IgG1 or IgG2 can be exemplified.

In the present invention, the term “anti-TROP2 antibody” refers to an antibody which binds specifically to TROP2 (TACSTD2: Tumor-associated calcium signal transducer 2; EGP-1), and preferably has an activity of internalization in TROP2-expressing cells by binding to TROP2.

Examples of the anti-TROP2 antibody include hTINA1-H1L1 (WO 2015/098099).

### 3. Production of Antibody-Drug Conjugate

A drug-linker intermediate for use in the production of the antibody-drug conjugate according to the present invention is represented by the following formula.

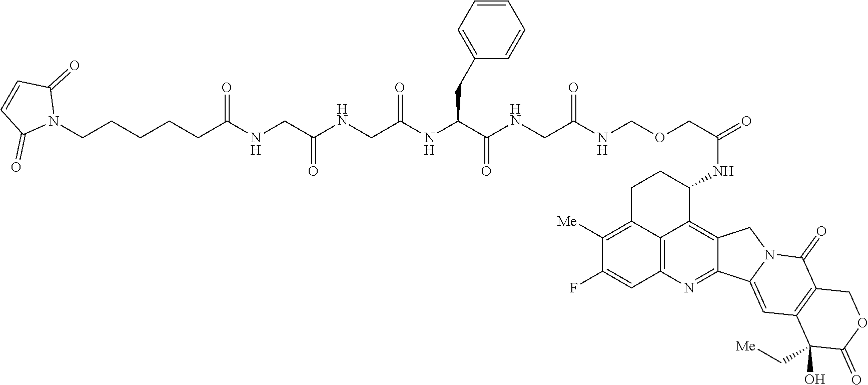 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl)hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl]amino}-2-oxoethoxy)methyl]glycinamide, and can be produced with reference to descriptions in WO 2014/057687, WO 2015/098099, WO 2015/115091, WO 2015/155998, WO 2019/044947, and so on.

The antibody-drug conjugate used in the present invention can be produced by reacting the above-described drug-linker intermediate and an anti-TROP2 antibody having a thiol group (alternatively referred to as a sulfhydryl group).

The anti-TROP2 antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl)phosphine hydrochloride (TCEP) per interchain disulfide within the antibody and reacting with the antibody in a buffer solution containing a chelating agent such as ethylenediamine tetraacetic acid (EDTA), an antibody having a sulfhydryl group with partially or completely reduced interchain disulfides within the antibody can be obtained.

Further, by using 2 to 20 molar equivalents of the drug-linker intermediate per the antibody having a sulfhydryl group, an antibody-drug conjugate in which 2 to 8 drug molecules are conjugated per antibody molecule can be produced.

The average number of conjugated drug molecules per antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the antibody and the drug-linker intermediate and calculation of the average number of conjugated drug molecules per antibody molecule of the antibody-drug conjugate can be performed with reference to descriptions in WO 2015/098099 and WO 2017/002776, for example.

In the present invention, the term “anti-TROP2 antibody-drug conjugate” refers to an antibody-drug conjugate such that the antibody in the antibody-drug conjugate according to the invention is an anti-TROP2 antibody.

The anti-TROP2 antibody is preferably an antibody comprising a heavy chain comprising CDRH1 consisting of an amino acid sequence represented by SEQ ID NO: 3 [=an amino acid sequence consisting of amino acid residues 50 to 54 of SEQ ID NO: 1], CDRH2 consisting of an amino acid sequence represented by SEQ ID NO: 4 [=an amino acid sequence consisting of amino acid residues 69 to 85 of SEQ ID NO: 1], and CDRH3 consisting of an amino acid sequence represented by SEQ ID NO: 5 [=an amino acid sequence consisting of amino acid residues 118 to 129 of SEQ ID NO: 1], and a light chain comprising CDRL1 consisting of an amino acid sequence represented by SEQ ID NO: 6 [=an amino acid sequence consisting of amino acid residues 44 to 54 of SEQ ID NO: 2], CDRL2 consisting of an amino acid sequence represented by SEQ ID NO: 7 [=an amino acid sequence consisting of amino acid residues 70 to 76 of SEQ ID NO: 2], and CDRL3 consisting of an amino acid sequence represented by SEQ ID NO: 8 [=an amino acid sequence consisting of amino acid residues 109 to 117 of SEQ ID NO: 2],
more preferably an antibody comprising a heavy chain comprising a heavy chain variable region consisting of an amino acid sequence represented by SEQ ID NO: 9 [=an amino acid sequence consisting of amino acid residues 20 to 140 of SEQ ID NO: 1], and a light chain comprising a light chain variable region consisting of an amino acid sequence represented by SEQ ID NO: 10 [=an amino acid sequence consisting of amino acid residues 21 to 129 of SEQ ID NO: 2], and
even more preferably an antibody comprising a heavy chain consisting of an amino acid sequence represented by SEQ ID NO: 12 [=an amino acid sequence consisting of amino acid residues 20 to 470 of SEQ ID NO: 1] and a light chain consisting of an amino acid sequence represented by SEQ ID NO: 13 [=amino acid residues 21 to 234 of SEQ ID NO: 2], or an antibody comprising a heavy chain consisting of an amino acid sequence represented by SEQ ID NO: 11 [=an amino acid sequence consisting of amino acid residues 20 to 469 of SEQ ID NO: 1] and a light chain consisting of an amino acid sequence represented by SEQ ID NO: 13 [=amino acid residues 21 to 234 of SEQ ID NO: 2].

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in WO 2015/098099 and WO 2017/002776.

In preferred embodiments, the anti-TROP2 antibody-drug conjugate is datopotamab deruxtecan (DS-1062).

### 4. ATR Inhibitor

In the present disclosure, the term “ATR inhibitor” refers to an agent that inhibits ATR (ataxia telangiectasia and rad3-related kinase). The ATR inhibitor in the present disclosure may selectively inhibit the kinase ATR, or may non-selectively inhibit ATR and inhibit also kinase(s) other than ATR. The ATR inhibitor in the present disclosure is not particularly limited as long as it is an agent that has the described characteristics, and preferred examples thereof can include those disclosed in WO2011/154737.

Other examples of ATR inhibitors which may be used according to the present disclosure are berzosertib (M6620; VX-970), M4344 (VX-803), BAY-1895344, ETP-46464, and VE-821.

Preferably, the ATR inhibitor in the present disclosure inhibits ATR selectively.

According to preferred embodiments of the ATR inhibitor used in the present disclosure, the ATR inhibitor is a compound represented by the following formula (I):

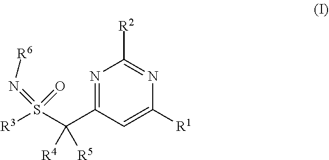

wherein:
R¹ is selected from morpholin-4-yl and 3-methylmorpholin-4-yl;
R² is

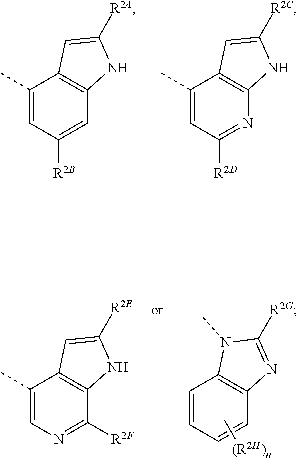

n is 0 or 1;
R^{2A}, R^{2C}, R^{2E} and R^{2F} each independently are hydrogen or methyl;
R^{2B} and R^{2D} each independently are hydrogen or methyl;
R^{2G} is selected from —NHR⁷ and —NHCOR⁸;
R^{2H} is fluoro;
R³ is methyl;
R⁴ and R⁵ are each independently hydrogen or methyl, or R⁴ and R⁵ together with the atom to which they are attached form Ring A;
Ring A is a C₃₋₆cycloalkyl or a saturated 4-6 membered heterocyclic ring containing one heteroatom selected from O and N;
R⁶ is hydrogen;
R⁷ is hydrogen or methyl; and
R⁸ is methyl,
or a pharmaceutically acceptable salt thereof.

In preferred embodiments, the ATR inhibitor is a compound represented by formula (I) wherein:
R¹ is 3-methylmorpholin-4-yl;
R² is

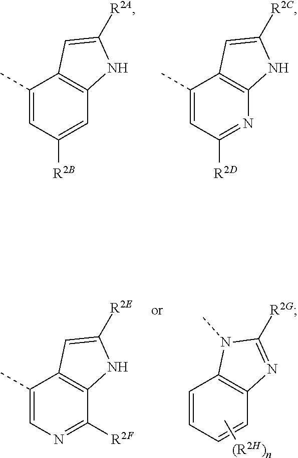

n is 0 or 1;
R^{2A}, R^{2C}, R^{2E} and R^{2F} each independently are hydrogen or methyl;
R^{2B} and R^{2D} each independently are hydrogen or methyl;
R^{2G} is selected from —NH₂, —NHMe and —NHCOMe;
R^{2H} is fluoro;
R³ is methyl;
R⁴ and R⁵ are each independently hydrogen or methyl, or R⁴ and R⁵ together with the atom to which they are attached form Ring A;
Ring A is a C₃₋₆cycloalkyl or a saturated 4-6 membered heterocyclic ring containing one heteroatom selected from O and N; and
R⁶ is hydrogen,
or a pharmaceutically acceptable salt thereof.

Additional embodiments of the ATR inhibitor are compounds of formula (I), and pharmaceutically acceptable salts thereof, in which Ring A, n, R¹, R², R⁴, R⁵, R⁶, R⁷ and R⁸ are defined as follows. Such specific substituents may be used, where appropriate, with any of the definitions, claims or embodiments defined herein.

n

In one embodiment n is 0.

In another embodiment n is 1.

### R¹

In one embodiment, R¹ is selected from morpholin-4-yl and 3-methylmorpholin-4-yl.

In a further embodiment, R¹ is 3-methylmorpholin-4-yl.

In a further embodiment, R¹ is

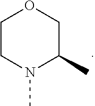

In a further embodiment, R¹ is

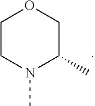

### R²

In one embodiment R² is

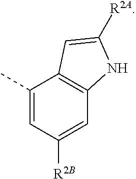

In another embodiment R² is

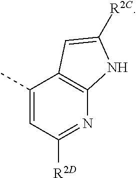

In another embodiment R² is

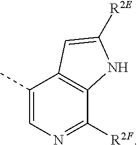

In another embodiment R² is

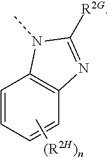

### R^{2A}

In one embodiment R^{2A} is hydrogen.

### R^{2B}

In one embodiment R^{2B} is hydrogen.

### R^{2C}

In one embodiment R^{2C} is hydrogen.

### R^{2D}

In one embodiment R^{2D} is hydrogen.

### R^{2E}

In one embodiment R^{2E} is hydrogen.

### R^{2F}

In one embodiment R^{2F} is hydrogen.

### R^{2G}

In one embodiment R^{2G} is selected from —NHR⁷ and —NHCOR⁸.

In another embodiment R^{2G} is —NHR⁷.

In another embodiment R^{2G} is —NHCOR⁸.

In another embodiment R^{2G} is selected from —NH₂, —NHMe and —NHCOMe.

In another embodiment of the disclosure R^{2G} is —NH₂.

In another embodiment R^{2G} is —NHMe.

In another embodiment R^{2G} is —NHCOMe.

R⁴ and R⁵

In one embodiment R⁴ and R⁵ are hydrogen.

In another embodiment R⁴ and R⁵ are methyl.

In another embodiment R⁴ and R⁵ together with the atom to which they are attached form Ring A.

### Ring A

In one embodiment Ring A is a C₃₋₆cycloalkyl or a saturated 4-6 heterocyclic ring containing one heteroatom selected from O and N.

In another embodiment Ring A is a cyclopropyl, cyclobutyl, cyclopentyl, oxetanyl, tetrahydrofuryl, tetrahydropyranyl, azetidinyl, pyrrolidinyl or piperidinyl ring.

In another embodiment Ring A is a cyclopropyl, cyclobutyl, cylopentyl, tetrahydropyranyl or piperidinyl ring.

In another embodiment Ring A is a cyclopropyl, cylopentyl, tetrahydropyranyl or piperidinyl ring.

In another embodiment Ring A is a cyclopropyl, tetrahydropyranyl or piperidinyl ring.

In another embodiment Ring A is a cyclopropyl or tetrahydropyranyl ring.

In another embodiment Ring A is a piperidinyl ring.

In another embodiment Ring A is a tetrahydropyranyl ring.

In another embodiment Ring A is a cyclopropyl ring.

### R⁶

In one embodiment R⁶ is hydrogen.

### R⁷

In one embodiment R⁷ is hydrogen or methyl.

In another embodiment R⁷ is methyl.

In another embodiment R⁷ is hydrogen.

### R⁸

In one embodiment R¹² is methyl.

In one embodiment of compounds of formula (I), or a pharmaceutically acceptable salt thereof: R¹ is selected from morpholin-4-yl and 3-methylmorpholin-4-yl;
n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is selected from —NHR⁷ and —NHCOR⁸;
R^{2H} is fluoro;
R³ is methyl;
R⁴ and R⁵ together with the atom to which they are attached form Ring A;
Ring A is a C₃₋₆cycloalkyl or a saturated 4-6 heterocyclic ring containing one heteroatom selected from O and N;
R⁶ is hydrogen;
R⁷ is hydrogen or methyl; and
R⁸ is methyl.

In another embodiment:
R¹ is selected from morpholin-4-yl and 3-methylmorpholin-4-yl;
n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is selected from —NH₂, —NHMe and —NHCOMe;
R^{2H} is fluoro;
R³ is methyl;
R⁴ and R⁵ together with the atom to which they are attached form Ring A;
Ring A is a C₃₋₆cycloalkyl or a saturated 4-6 heterocyclic ring containing one heteroatom selected from O and N; and
R⁶ is hydrogen.

In another embodiment:
R¹ is selected from morpholin-4-yl and 3-methylmorpholin-4-yl;
n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is selected from —NHR⁷ and —NHCOR⁸;
R^{2H} is fluoro;
R³ is methyl;
R⁴ and R⁵ together with the atom to which they are attached form Ring A;
Ring A is a cyclopropyl, cyclobutyl, cyclopentyl, oxetanyl, tetrahydrofuryl, tetrahydropyranyl, azetidinyl, pyrrolidinyl or piperidinyl ring;
R⁶ is hydrogen;
R⁷ is hydrogen or methyl; and
R⁸ is methyl.

In another embodiment:
R¹ is selected from morpholin-4-yl and 3-methylmorpholin-4-yl;
n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is selected from —NH₂, —NHMe and —NHCOMe;
R^{2H} is fluoro;
R³ is methyl;
R⁴ and R⁵ together with the atom to which they are attached form Ring A;
Ring A is a cyclopropyl, cyclobutyl, cyclopentyl, oxetanyl, tetrahydrofuryl, tetrahydropyranyl, azetidinyl, pyrrolidinyl or piperidinyl ring; and
R⁶ is hydrogen.

In another embodiment, compounds of formula (I) are compounds of formula (Ia):

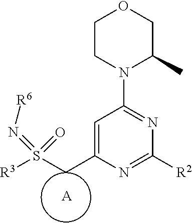

(Ia)
or a pharmaceutically acceptable salt thereof, in which: Ring A is a cyclopropyl, tetrahydropyranyl or piperidinyl ring;
R² is

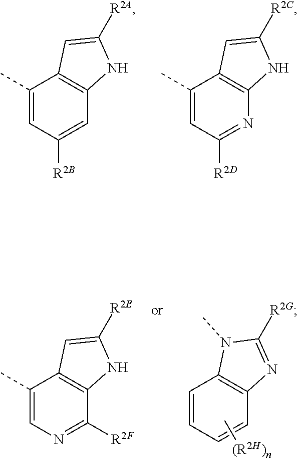

n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is selected from —NHR⁷ and —NHCOR⁸;
R^{2H} is fluoro;
R³ is a methyl group;
R⁶ is hydrogen;
R⁷ is hydrogen or methyl; and
R⁸ is methyl.

In another embodiment, compounds of formula (I) are compounds of formula (Ia) or a pharmaceutically acceptable salt thereof, in which:
Ring A is a cyclopropyl, tetrahydropyranyl or piperidinyl ring;
R² is

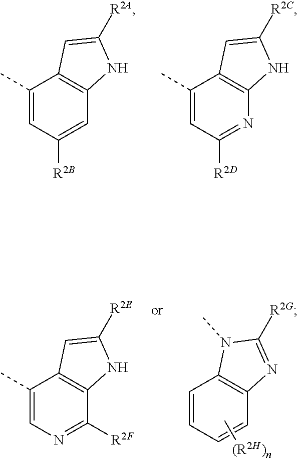

n is 0 or 1;
R² is hydrogen;
R^{2A} is hydrogen;
R^{2C} is hydrogen;
R^{2C} is hydrogen;
R^{2D} is hydrogen;
R^{2F} is hydrogen;
R^{2G} is selected from —NH₂, —NHMe and —NHCOMe;
R^{2H} is fluoro;
R³ is a methyl group; and
R⁶ is hydrogen.

In another embodiment, compounds of formula (I) are compounds of formula (Ia) or a pharmaceutically acceptable salt thereof, in which:
Ring A is a cyclopropyl, tetrahydropyranyl or piperidinyl ring;
R² is

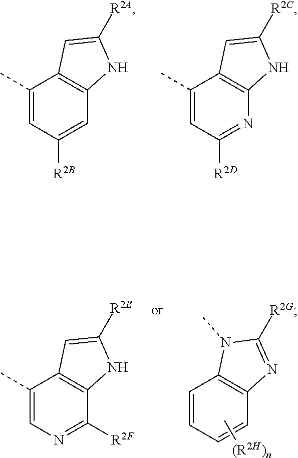

n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is —NHR⁷;
R^{2H} is fluoro;
R³ is a methyl group;
R⁶ is hydrogen; and
R⁷ is hydrogen.

In another embodiment, compounds of formula (I) are compounds of formula (Ia) or a pharmaceutically acceptable salt thereof, in which:
Ring A is a cyclopropyl ring;
R² is

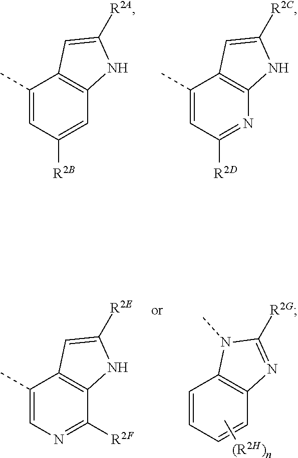

n is 0;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is —NHR⁷;
R^{2H} is fluoro;
R³ is a methyl group;
R⁶ is hydrogen; and
R⁷ is methyl.

In other embodiments, the ATR inhibitor used in the disclosure is a compound selected from:
4-{4-[(3R)-3-methylmorpholin-4-yl]-6-[((R)-S-methylsulfonimidoyl)methyl]pyrimidin-2-yl}-1H-pyrrolo[2,3-b]pyridine;
4-{4-[(3R)-3-methylmorpholin-4-yl]-6-[1-((S)-S-methylsulfonimidoyl)cyclopropyl]pyrimidin-2-yl}-1H-pyrrolo[2,3-b]pyridine;
4-{4-[(3R)-3-methylmorpholin-4-yl]-6-[1-((R)-S-methylsulfonimidoyl)cyclopropyl]pyrimidin-2-yl}-1H-pyrrolo[2,3-b]pyridine;
N-methyl-1-{4-[(3R)-3-methylmorpholin-4-yl]-6-[1-((R)-S-methylsulfonimidoyl)cyclopropyl]pyrimidin-2-yl}-1H-benzimidazol-2-amine;
N-methyl-1-{4-[(3R)-3-methylmorpholin-4-yl]-6-[1-((S)-S-methylsulfonimidoyl)cyclopropyl]pyrimidin-2-yl}-1H-benzimidazol-2-amine;
4-{4-[(3R)-3-methylmorpholin-4-yl]-6-[1-((R)-S-methylsulfonimidoyl)cyclopropyl]pyrimidin-2-yl}-1H-indole;
4-{4-[(3R)-3-methylmorpholin-4-yl]-6-[1-((S)-S-methylsulfonimidoyl)cyclopropyl]pyrimidin-2-yl}-1H-indole;
1-{4-[(3R)-3-methylmorpholin-4-yl]-6-[1-((R)-S-methylsulfonimidoyl)cyclopropyl]pyrimidin-2-yl}-1H-benzimidazol-2-amine;
1-{4-[(3R)-3-methylmorpholin-4-yl]-6-[1-((S)-S-methylsulfonimidoyl)cyclopropyl]pyrimidin-2-yl}-1H-benzimidazol-2-amine;
4-fluoro-N-methyl-1-{4-[(3R)-3-methylmorpholin-4-yl]-6-[1-((R)-S-methylsulfonimidoyl)cyclopropyl]pyrimidin-2-yl}-1H-benzimidazol-2-amine;
4-fluoro-N-methyl-1-{4-[(3R)-3-methylmorpholin-4-yl]-6-[1-((S)-S-methylsulfonimidoyl)cyclopropyl]pyrimidin-2-yl}-1H-benzimidazol-2-amine;
4-{4-[(3R)-3-methylmorpholin-4-yl]-6-[1-(S-methylsulfonimidoyl)cyclopropyl]pyrimidin-2-yl}-1H-pyrrolo[2,3-c]pyridine;
N-methyl-1-{4-[1-methyl-1-((S)-S-methylsulfonimidoyl)ethyl]-6-[(3R)-3-methylmorpholin-4-yl]pyrimidin-2-yl}-1H-benzimidazol-2-amine;
N-methyl-1-{4-[i-methyl-1-((R)-S-methylsulfonimidoyl)ethyl]-6-[(3R)-3-methylmorpholin-4-yl]pyrimidin-2-yl}-1H-benzimidazol-2-amine;
N-methyl-1-{4-[(3R)-3-methylmorpholin-4-yl]-6-[4-((S)-S-methylsulfonimidoyl)tetrahydro-2H-pyran-4-yl]pyrimidin-2-yl}-1H-benzimidazol-2-amine;
N-methyl-1-{4-[(3R)-3-methylmorpholin-4-yl]-6-[4-((R)-S-methylsulfonimidoyl)tetrahydro-2H-pyran-4-yl]pyrimidin-2-yl}-1H-benzimidazol-2-amine;
4-{4-[(3R)-3-methylmorpholin-4-yl]-6-[4-((S)-S-methylsulfonimidoyl)tetrahydro-2H-pyran-4-yl]pyrimidin-2-yl}-1H-indole;
4-fluoro-N-methyl-1-{4-[i-methyl-1-((S)-S-methylsulfonimidoyl)ethyl]-6-[(3R)-3-methylmorpholin-4-yl]pyrimidin-2-yl}-1H-benzimidazol-2-amine;
4-fluoro-N-methyl-1-{4-[i-methyl-1-((R)-S-methylsulfonimidoyl)ethyl]-6-[(3R)-3-methylmorpholin-4-yl]pyrimidin-2-yl}-1H-benzimidazol-2-amine;
6-fluoro-N-methyl-1-{4-[1-methyl-1-((R)-S-methylsulfonimidoyl)ethyl]-6-[(3R)-3-methylmorpholin-4-yl]pyrimidin-2-yl}-1H-benzimidazol-2-amine;
5-fluoro-N-methyl-1-{4-[1-methyl-1-((R)-S-methylsulfonimidoyl)ethyl]-6-[(3R)-3-methylmorpholin-4-yl]pyrimidin-2-yl}-1H-benzimidazol-2-amine;
5-fluoro-N-methyl-1-{4-[1-methyl-1-((S)-S-methylsulfonimidoyl)ethyl]-6-[(3R)-3-methylmorpholin-4-yl]pyrimidin-2-yl}-1H-benzimidazol-2-amine;
6-fluoro-N-methyl-1-{4-[1-methyl-1-((S)-S-methylsulfonimidoyl)ethyl]-6-[(3R)-3-methylmorpholin-4-yl]pyrimidin-2-yl}-1H-benzimidazol-2-amine;
6-fluoro-N-methyl-1-{4-[(3R)-3-methylmorpholin-4-yl]-6-[1-((R)-S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((R)-S-methylsulfonimidoyl)cyclopropyl]pyrimidin-2-yl}-1H-benzimidazol-2-amine;
5-fluoro-N-methyl-1-{4-[(3R)-3-methylmorpholin-4-yl]-6-[1-((S)-S-methylsulfonimidoyl)cyclopropyl]pyrimidin-2-yl}-1H-benzimidazol-2-amine;
6-fluoro-N-methyl-1-{4-[(3R)-3-methylmorpholin-4-yl]-6-[1-((S)-S-methylsulfonimidoyl)cyclopropyl]pyrimidin-2-yl}-1H-benzimidazol-2-amine,
   and pharmaceutically acceptable salts thereof.

In other embodiments, the ATR inhibitor used in the disclosure is a compound selected from:
4-{4-[(3R)-3-methylmorpholin-4-yl]-6-[(R)-(S-methylsulfonimidoyl)methyl]pyrimidin-2-yl}-1H-pyrrolo[2,3-b]pyridine;
4-{4-[(3R)-3-methylmorpholin-4-yl]-6-[1-((S)-S-methylsulfonimidoyl)cyclopropyl]pyrimidin-2-yl}-1H-pyrrolo[2,3-b]pyridine;
4-{4-[(3R)-3-methylmorpholin-4-yl]-6-[1-((R)-S-methylsulfonimidoyl)cyclopropyl]pyrimidin-2-yl}-1H-pyrrolo[2,3-b]pyridine;
N-methyl-1-{4-[(3R)-3-methylmorpholin-4-yl]-6-[1-(R)-(S-methylsulfonimidoyl)cyclopropyl]pyrimidin-2-yl}-1H-benzimidazol-2-amine;
N-methyl-1-{4-[(3R)-3-methylmorpholin-4-yl]-6-[1-(S)-(S-methylsulfonimidoyl)cyclopropyl]pyrimidin-2-yl}-1H-benzimidazol-2-amine,
   and pharmaceutically acceptable salts thereof.

In a preferred embodiment the ATR inhibitor used in the disclosure is the compound AZD6738, 4-{4-[(3R)-3-methylmorpholin-4-yl]-6-[l-((R)-S-methylsulfonimidoyl)cyclopropyl]pyrimidin-2-yl}-1H-pyrrolo[2,3-b]pyridine, represented by the following formula:

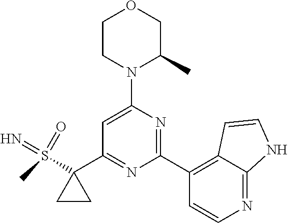 [SMILES: C[C@@H]1COCCN1c1cc(C2([S@](C)(=N)=O)CC2)nc(-c2ccnc3[nH]ccc23)n1]

or a pharmaceutically acceptable salt thereof.

ATR inhibitors such as compounds of formula (I), including AZD6738, may be prepared by methods known in the art such as disclosed in WO2011/154737.

### 5. Combination of Antibody-Drug Conjugate and ATR Inhibitor

In a first combination embodiment of the disclosure, the anti-TROP2 antibody-drug conjugate which is combined with the ATR inhibitor is an antibody-drug conjugate in which a drug-linker represented by the following formula:

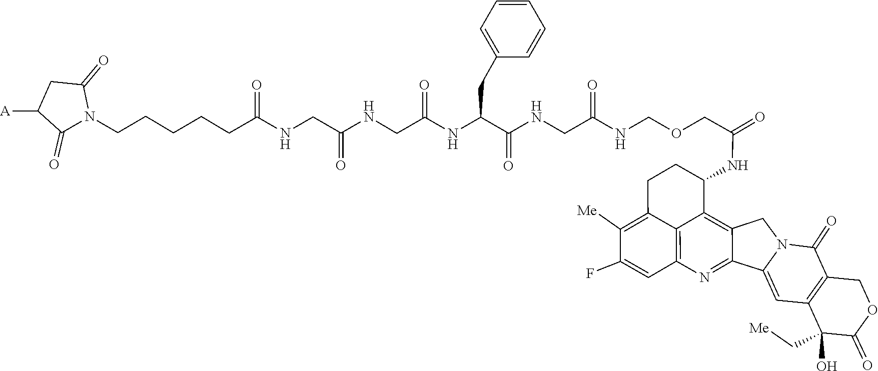

wherein A represents the connecting position to an antibody, is conjugated to an anti-TROP2 antibody via a thioether bond.

In another combination embodiment, the anti-TROP2 antibody-drug conjugate as defined above for the first combination embodiment is combined with an ATR inhibitor which is a compound represented by the following formula (I):

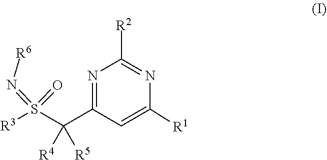

wherein:
R¹ is selected from morpholin-4-yl and 3-methylmorpholin-4-yl;
R² is

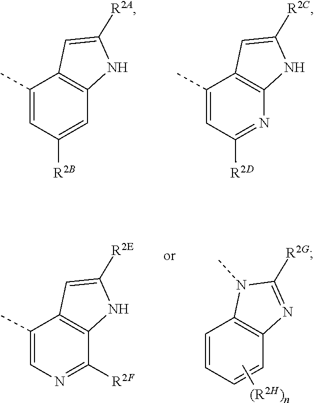

n is 0 or 1;
R^{2A}, R^{2C}, R^{2E} and R^{2F} each independently are hydrogen or methyl;
R^{2B} and R^{2D} each independently are hydrogen or methyl;
R^{2G} is selected from —NHR⁷ and —NHCOR⁸;
R^{2H} is fluoro;
R³ is methyl;
R⁴ and R⁵ are each independently hydrogen or methyl, or R⁴ and R⁵ together with the atom to which they are attached form Ring A;
Ring A is a C₃₋₆cycloalkyl or a saturated 4-6 membered heterocyclic ring containing one heteroatom selected from O and N;
R⁶ is hydrogen;
R⁷ is hydrogen or methyl;
R⁸ is methyl,
   or a pharmaceutically acceptable salt thereof.

In another combination embodiment, the anti-TROP2 antibody-drug conjugate as defined above is combined with an ATR inhibitor which is a compound represented by formula (I) as defined above wherein, in formula (I), R⁴ and R⁵ together with the atom to which they are attached form Ring A, and Ring A is a C₃₋₆cycloalkyl or a saturated 4-6 heterocyclic ring containing one heteroatom selected from O and N.

In another combination embodiment, the anti-TROP2 antibody-drug conjugate as defined above is combined with an ATR inhibitor as defined above wherein, in formula (I), R⁴ and R⁵ together with the atom to which they are attached form Ring A, and Ring A is a cyclopropyl, tetrahydropyranyl or piperidinyl ring.

In another combination embodiment, the anti-TROP2 antibody-drug conjugate as defined above is combined with an ATR inhibitor as defined above wherein, in formula (I), R^{2A} is hydrogen; R^{2B} is hydrogen; R^{2C} is hydrogen; R^{2D} is hydrogen; R^{2E} is hydrogen; and R^{2F} is hydrogen.

In another combination embodiment, the anti-TROP2 antibody-drug conjugate as defined above is combined with an ATR inhibitor as defined above wherein, in formula (I), R¹ is 3-methylmorpholin-4-yl.

In another combination embodiment, the anti-TROP2 antibody-drug conjugate as defined above is combined with an ATR inhibitor as defined above, wherein the compound of formula (I) is a compound of formula (Ia):

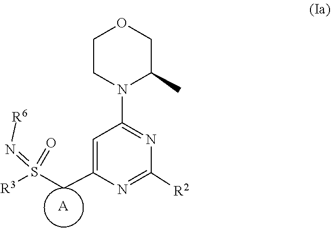

or a pharmaceutically acceptable salt thereof.

In another combination embodiment, the anti-TROP2 antibody-drug conjugate as defined above is combined with an ATR inhibitor as defined above wherein the compound of formula (I) is a compound of formula (Ia) wherein, in formula (Ia):
Ring A is cyclopropyl ring;
R² is

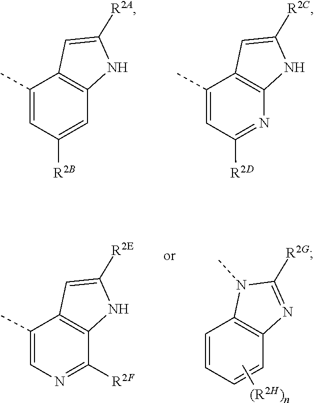

n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is —NHR⁷;
R^{2H} is fluoro;
R³ is a methyl group;
R⁶ is hydrogen; and
R⁷ is hydrogen or methyl.

In another combination embodiment, the anti-TROP2 antibody-drug conjugate as defined above is combined with an ATR inhibitor as defined above, wherein the ATR inhibitor is AZD6738 represented by the following formula:

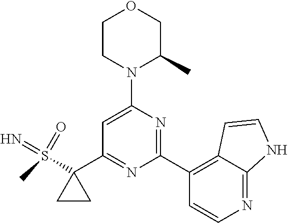 [SMILES: C[C@@H]1COCCN1c1cc(C2([S@](C)(=N)=O)CC2)nc(-c2ccnc3[nH]ccc23)n1]

or a pharmaceutically acceptable salt thereof.

In an embodiment of each of the combination embodiments described above, the anti-TROP2 antibody comprises a heavy chain comprising CDRH1 consisting of an amino acid sequence represented by SEQ ID NO: 3 [=amino acid residues 50 to 54 of SEQ ID NO: 1], CDRH2 consisting of an amino acid sequence represented by SEQ ID NO: 4 [=amino acid residues 69 to 85 of SEQ ID NO: 1] and CDRH3 consisting of an amino acid sequence represented by SEQ ID NO: 5 [=amino acid residues 118 to 129 of SEQ ID NO: 1], and a light chain comprising CDRL1 consisting of an amino acid sequence represented by SEQ ID NO: 6 [=amino acid residues 44 to 54 of SEQ ID NO: 2], CDRL2 consisting of an amino acid sequence represented by SEQ ID NO: 7 [=amino acid residues 70 to 76 of SEQ ID NO: 2] and CDRL3 consisting of an amino acid sequence represented by SEQ ID NO: 8 [=amino acid residues 109 to 117 of SEQ ID NO: 2]. In another embodiment of each of the combination embodiments described above, the anti-TROP2 antibody comprises a heavy chain comprising a heavy chain variable region consisting of an amino acid sequence represented by SEQ ID NO: 9 [=amino acid residues 20 to 140 of SEQ ID NO: 1] and a light chain comprising a light chain variable region consisting of an amino acid sequence represented by SEQ ID NO: 10 [=amino acid residues 21 to 129 of SEQ ID NO: 2]. In another embodiment of each of the combination embodiments described above, the anti-TROP2 antibody comprises a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]. In another embodiment of each of the combination embodiments described above, the anti-TROP2 antibody comprises a heavy chain consisting of an amino acid sequence represented by SEQ ID NO: 11 [=amino acid residues 20 to 469 of SEQ ID NO: 1] and a light chain consisting of an amino acid sequence represented by SEQ ID NO: 13 [=amino acid residues 21 to 234 of SEQ ID NO: 2].

In a particularly preferred combination embodiment of the disclosure, the anti-TROP2 antibody-drug conjugate is datopotamab deruxtecan (DS-1062) and the ATR inhibitor is the compound represented by the following formula:

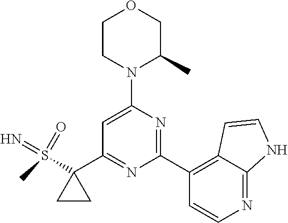 [SMILES: C[C@@H]1COCCN1c1cc(C2([S@](C)(=N)=O)CC2)nc(-c2ccnc3[nH]ccc23)n1]

also identified as AZD6738.

### 6. Therapeutic Combined Use and Method

Described in the following are a pharmaceutical product and a therapeutic use and method wherein the anti-TROP2 antibody-drug conjugate according to the present disclosure and an ATR inhibitor are administered in combination.

The pharmaceutical product and therapeutic use and method of the present disclosure may be characterized in that the anti-TROP2 antibody-drug conjugate and the ATR inhibitor are separately contained as active components in different formulations, and are administered simultaneously or at different times.

In the pharmaceutical product and therapeutic method of the present disclosure, a single ATR inhibitor used in the present disclosure can be administered in combination with the anti-TROP2 antibody-drug conjugate, or two or more different ATR inhibitors can be administered in combination with the antibody-drug conjugate.

The pharmaceutical product and therapeutic method of the present disclosure can be used for treating cancer, and can be preferably used for treating at least one cancer selected from the group consisting of breast cancer (including triple negative breast cancer and hormone receptor (HR)-positive, HER2-negative breast cancer), lung cancer (including small cell lung cancer and non-small cell lung cancer), colorectal cancer (also called colon and rectal cancer, and including colon cancer and rectal cancer), gastric cancer (also called gastric adenocarcinoma), esophageal cancer, head-and-neck cancer (including salivary gland cancer and pharyngeal cancer), esophagogastric junction adenocarcinoma, biliary tract cancer (including bile duct cancer), Paget's disease, pancreatic cancer, ovarian cancer, uterine carcinosarcoma, urothelial cancer, prostate cancer, bladder cancer, gastrointestinal stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma, and can be more preferably used for treating at least one cancer selected from the group consisting of breast cancer (preferably triple negative breast cancer and hormone receptor (HR)-positive, HER2-negative breast cancer), lung cancer (preferably non-small cell lung cancer, including non-small cell lung cancer with actionable genomic alterations and non-small cell lung cancer without actionable genomic alterations, wherein the actionable genomic alterations include EGFR, ALK, ROS1, NTRK, BRAF, RET, and MET exon 14 skipping), colorectal cancer, gastric cancer, pancreatic cancer, ovarian cancer, prostate cancer, and kidney cancer. Furthermore, the pharmaceutical product and therapeutic method of the present disclosure can preferably be used for treating cancer that is deficient in Homologous Recombination (HR) dependent DNA DSB repair activity, or cancer that is not deficient in Homologous Recombination (HR) dependent DNA DSB repair activity. Furthermore, the pharmaceutical product and therapeutic method of the present disclosure can preferably be used for treating cancer that exhibits resistance or refractoriness to a previous treatment with a PARP inhibitor (in particular a PARP inhibitor selected from olaparib, rucaparib, niraparib, talazoparib and veliparib). The presence or absence of TROP2 tumor markers can be determined, for example, by collecting tumor tissue from a cancer patient to prepare a formalin-fixed, paraffin-embedded (FFPE) specimen and subjecting the specimen to a test for gene products (proteins), for example, with an immunohistochemical (IHC) method, a flow cytometer, or Western blotting, or to a test for gene transcription, for example, with an in situ hybridization (ISH) method, a quantitative PCR method (q-PCR), or microarray analysis, or by collecting cell-free circulating tumor DNA (ctDNA) from a cancer patient and subjecting the ctDNA to a test with a method such as next-generation sequencing (NGS).

The pharmaceutical product and therapeutic method of the present disclosure can be preferably used for mammals, and can be more preferably used for humans.

The antitumor effect of the pharmaceutical product and therapeutic method of the present disclosure can be confirmed by, for example, generating a model in which cancer cells are transplanted to a test animal, and measuring reduction in tumor volume, life-prolonging effects due to applying the pharmaceutical product and therapeutic method of the present disclosure. Furthermore, comparison with the antitumor effect of single administration of each of the antibody-drug conjugate and the ATR inhibitor used in the present invention can provide confirmation of the combined effect of the antibody-drug conjugate and the ATR inhibitor used in the present disclosure.

In addition, the antitumor effect of the pharmaceutical product and therapeutic method of the present disclosure can be confirmed, in a clinical study, with the Response Evaluation Criteria in Solid Tumors (RECIST) evaluation method, WHO's evaluation method, Macdonald's evaluation method, measurement of body weight, and other methods; and can be determined by indicators such as Complete response (CR), Partial response (PR), Progressive disease (PD), Objective response rate (ORR), Duration of response (DoR), Progression-free survival (PFS), and Overall survival (OS).

The foregoing methods can provide confirmation of superiority in terms of the antitumor effect of the pharmaceutical product and therapeutic method of the present disclosure compared to existing pharmaceutical products and treatment methods for cancer therapy.

The pharmaceutical product and therapeutic method of the present disclosure can retard growth of cancer cells, suppress their proliferation, and further can kill cancer cells. These effects can allow cancer patients to be free from symptoms caused by cancer or can achieve an improvement in the QOL of cancer patients and attain a therapeutic effect by sustaining the lives of the cancer patients. Even if the pharmaceutical product and therapeutic method do not accomplish the killing of cancer cells, they can achieve higher QOL of cancer patients while achieving longer-term survival, by inhibiting or controlling the growth of cancer cells.

The pharmaceutical product of the present invention can be expected to exert a therapeutic effect by application as systemic therapy to patients, and additionally, by local application to cancer tissues.

The pharmaceutical product and therapeutic method of the present disclosure, in another aspect, provides for use as an adjunct in cancer therapy with ionizing radiation or other chemotherapeutic agents. For example, in the treatment of cancer, the treatment may comprise administering to a subject in need of treatment a therapeutically-effective amount of the pharmaceutical product, simultaneously or sequentially with ionizing radiation or other chemotherapeutic agents.

The pharmaceutical product and therapeutic method of the present disclosure can be used as adjuvant chemotherapy combined with surgery operation. The pharmaceutical product of the present disclosure may be administered for the purpose of reducing tumor size before surgical operation (referred to as preoperative adjuvant chemotherapy or neoadjuvant therapy), or may be administered for the purpose of preventing recurrence of tumor after surgical operation (referred to as postoperative adjuvant chemotherapy or adjuvant therapy).

In further aspects, the pharmaceutical product of the present disclosure may be used for the treatment of cancer which is deficient in Homologous Recombination (HR) dependent DNA DSB repair activity. The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_000051), RAD51 (NM_002875), RAD51L1 (NM_002877), RAD51C (NM_002876), RAD51L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE11A (NM_005590) and NBS1 (NM_002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001). A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells. The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535). BRCA1 and BRCA2 are known tumor suppressors whose wild-type alleles are frequently lost in tumors of heterozygous carriers (Jasin M., Oncogene, 21(58), 8981-93 (2002); Tutt, et al., Trends Mol Med., 8 (12), 571-6, (2002)). The association of BRCA1 and/or BRCA2 mutations with breast cancer is well-characterised in the art (Radice, P. J., Exp Clin Cancer Res., 21(3 Suppl), 9-12 (2002)). Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast, ovary, pancreas, prostate, hematological, gastrointestinal and lung cancer. In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Chappnis, P. O. and Foulkes, W. O., Cancer Treat Res, 107, 29-59 (2002); Janatova M., et al., Neoplasma, 50(4), 246-505 (2003); Jancarkova, N., Ceska Gynekol., 68{1), 11-6 (2003)). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The pharmaceutical product of the present disclosure may be administered as a pharmaceutical composition containing at least one pharmaceutically suitable ingredient. The pharmaceutically suitable ingredient can be suitably selected and applied from formulation additives or the like that are generally used in the art, in accordance with the dosage, administration concentration or the like of the antibody-drug conjugate used in the present disclosure and the ATR inhibitor. For example, the antibody-drug conjugate used in the present disclosure can be administered as a pharmaceutical composition containing a buffer such as a histidine buffer, an excipient such as sucrose or trehalose, and a surfactant such as Polysorbate 80 or 20. The pharmaceutical product containing the antibody-drug conjugate used in the present disclosure can be preferably used as an injection, can be more preferably used as an aqueous injection or a lyophilized injection, and can be even more preferably used as a lyophilized injection. In the case that the pharmaceutical product containing the anti-TROP2 antibody-drug conjugate used in the present disclosure is an aqueous injection, it can be preferably diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified. In the case that the pharmaceutical product of the present disclosure is a lyophilized injection, it can be preferably dissolved in water for injection, subsequently a required amount can be diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified.

Examples of the administration route which may be used to administer the pharmaceutical product of the present disclosure include intravenous, intradermal, subcutaneous, intramuscular, and intraperitoneal routes, and preferably include an intravenous route.

The anti-TROP2 antibody-drug conjugate used in the present disclosure can be administered to a human once at intervals of 1 to 180 days, and can be preferably administered once a week, once every 2 weeks, once every 3 weeks, or once every 4 weeks, and can be even more preferably administered once every 3 weeks. Also, the antibody-drug conjugate used in the present invention can be administered at a dose of about 0.001 to 100 mg/kg, and can be preferably administered at a dose of 0.8 to 12.4 mg/kg, more preferably at a dose of 6 mg/kg. For example, the anti-TROP2 antibody-drug conjugate can be administered once every 3 weeks at a dose of 0.27 mg/kg, 0.5 mg/kg, 1.0 mg/kg, 2.0 mg/kg, 4.0 mg/kg, 6.0 mg/kg, or 8.0 mg/kg, and can be preferably administered once every 3 weeks at a dose of 6.0 mg/kg.

For example, a formulation of an ATR inhibitor compound of formula (I) intended for oral administration to humans will generally contain, for example, from 1 mg to 1000 mg of the active ingredient, compounded with an appropriate and convenient amount of excipients which may vary from about 5 to about 98 percent by weight of the total composition. For further information on Routes of Administration and Dosage Regimes, reference may be made to Chapter 25.3 in Volume 5 of Comprehensive Medicinal Chemistry (Corwin Hansch; Chairman of Editorial Board), Pergamon Press 1990.

The size of the dose required for the therapeutic treatment of a particular disease state will necessarily be varied depending on the subject treated, the route of administration and the severity of the illness being treated. A daily dose of the ATR inhibitor in the range of 0.1-50 mg/kg may be employed. For example, the ATR inhibitor is preferably administered daily for the first week, second week, and/or third week of a three week cycle, for example on days 3 to 17 of a three week cycle. For example, in the case that the ATR inhibitor used in the present disclosure is the compound AZD6738 or a pharmaceutically acceptable salt thereof, the ATR inhibitor can be preferably orally administered twice per day in a dose of 20 mg, 40 mg, 60 mg, 80 mg, 120 mg, 160 mg, 200 mg or 240 mg per administration.

### EXAMPLES

The present disclosure is specifically described in view of the examples shown below. However, the present disclosure is not limited to these. Further, it is by no means to be interpreted in a limited way.

### Example 1: Production of Anti-TROP2 Antibody-Drug Conjugate

In accordance with a production method described in WO 2015/098099 and WO 2017/002776 and using an anti-TROP2 antibody (an antibody comprising a heavy chain consisting of an amino acid sequence represented by SEQ ID NO: 12 [=amino acid residues 20 to 470 of SEQ ID NO: 1] and a light chain consisting of an amino acid sequence represented by SEQ ID NO: 13 [=amino acid residues 21 to 234 of SEQ ID NO: 2]), an anti-TROP2 antibody-drug conjugate in which a drug-linker represented by the following formula:

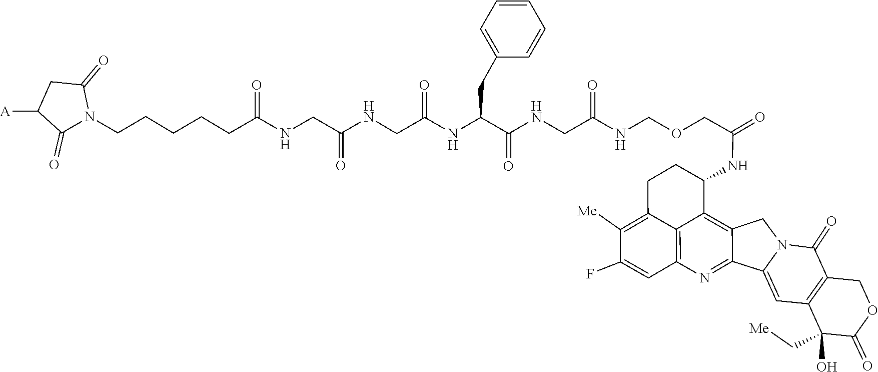

wherein A represents the connecting position to an antibody, is conjugated to the anti-TROP2 antibody via a thioether bond was produced (DS-1062: datopotamab deruxtecan). The DAR of the antibody-drug conjugate is ˜4.

### Example 2: Production of ATR Inhibitor

In accordance with a production method described in WO2011/154737), an ATR inhibitor of formula (I) is prepared. Specifically, 4-{4-[(3R)-3-methylmorpholin-4-yl]-6-[1-((R)-S-methylsulfonimidoyl)cyclopropyl]pyrimidin-2-yl}-1H-pyrrolo[2,3-b]pyridine:

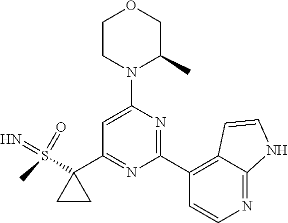 [SMILES: C[C@@H]1COCCN1c1cc(C2([S@](C)(=N)=O)CC2)nc(-c2ccnc3[nH]ccc23)n1]

can be prepared according to Example 2.02 of WO2011/154737.

### Example 3: Antitumor Test

Combination of antibody-drug conjugate DS-1062 (datopotamab deruxtecan) with AZD6738 (4-{4-[(3R)-3-Methylmorpholin-4-yl]-6-[1-((R)-S-methylsulfonimidoyl)cyclopropyl]pyrimidin-2-yl}-1H-pyrrolo[2,3-b]pyridine)

### Method:

A high-throughput combination screen was run, in which 6 lung and 5 breast cancer cell lines (Table 1) were treated with combinations of DS-1062 and AZD6738 (ATR inhibitor).

TABLE 1

| Cell Line | Cancer Type |
|---|---|
| NCI-H1975 | Lung |
| NCI-H1650 | Lung |
| NC-H322 | Lung |
| HCC2935 | Lung |
| NCI-H3255 | Lung |
| CALU3 | Lung |
| HCC70 | Breast |
| HCC1187 | Breast |
| HCC1806 | Breast |
| MDA-MB-468 | Breast |
| HCC38 | Breast |

The readout of the screen was a 7-day CellTiter-Glo cell viability assay, conducted as a 6×6 dose response matrix (5-point log serial dilution for DS-1062, and half log serial dilution for AZD6738). Maximum concentration was 3 μM for AZD6738, and 10 μg/ml for DS-1062. In addition, exatecan (DNA topoisomerase I inhibitor) was also screened in parallel with AZD6738, to help deconvolute the mechanism of action of effective combinations. Combination activity was assessed based on a combination of the Combination Emax and Loewe synergy scores.

### Results:

Results for the DS-1062+AZD6738 combination are shown for TROP2-expressing lung cancer cell lines (NCIH1650, NCI-H322, NCI-H3255, HCC2935) in FIGS. 12A and 12B and Table 2, and for TROP2-expressing breast cancer cell lines (HCC70, HCC1806, MDA-MB-468, HCC38) in FIGS. 13A and 13B and Table 3.

FIGS. 12A and 13A show matrices of measured cell viability signals. X axes represent drug A (DS-1062), and Y axes represent drug B (AZD6738). Values in the box represent the ratio of cells treated with drug A+B compared to DMSO control at day 7. All values are normalised to cell viability values at day 0. Values between 0 and 100 represent % growth inhibition and values above 100 represent cell death.

FIGS. 12B and 13B show Loewe excess matrices. Values in the box represent excess values calculated by the Loewe additivity model.

Tables 2 and 3 show HSA and Loewe additivity scores and Combination Emax:

TABLE 2
DS-1062 + AZD6738 Combination
Results in Lung Cancer Cell Lines

| | NCI- | NCI- | NCI- | |
|---|---|---|---|---|
| Cell line | H1650 | H322 | H3255 | HCC2935 |
| HSA synergy | 9.72 | 6.28 | 12.01 | 14.17 |
| score | | | | |
| Loewe synergy | 9.56 | 5.49 | 12.01 | 12.86 |
| score | | | | |
| Combination | 166 | 115 | 178 | 165 |
| Emax | | | | |

TABLE 3
DS-1062 + AZD6738 Combination Results in Breast
Cancer Cell Lines

| | | | MDA- | |
|---|---|---|---|---|
| Cell line | HCC70 | HCC1806 | MB-468 | HCC38 |
| HSA synergy | 9.73 | 11.37 | 18.03 | 6.85 |
| score | | | | |
| Loewe synergy | 9.72 | 9.58 | 17.09 | 6.46 |
| score | | | | |
| Combination | 189 | 165 | 181 | 148 |
| Emax | | | | |

### Notes:

Loewe Dose Additivity predicts the expected response if the two compounds act on the same molecular target by means of the same mechanism. It calculates additivity based on the assumption of zero interaction between the compounds and it is independent from the nature of the dose-response relationship.
HSA (Highest Single Agent) [Berenbaum 1989]quantifies the higher of the two single compound effects at their corresponding concentrations. The combined effect is compared with the effect of each single agent at the concentration used in the combination. Excess over the highest single agent effect indicates cooperativity. HSA does not require the compounds to affect the same target.
Excess Matrix: For each well in the concentration matrix, the measured or fitted values are compared to the predicted non-synergistic values for each concentration pair. The predicted values are determined by the chosen model. Differences between the predicted and observed values may indicate synergy or antagonism, and are shown in the Excess Matrix. Excess Matrix values are summarized by the combination scores Excess Volume and Synergy Score.
Combination Emax: The maximum anti-proliferative effect observed in the combination matrix tested. All values are normalised to cell viability values at day 0. Values between 0 and 100 represent % growth inhibition and values above 100 represent cell death.

FIG. 14 shows combination Emax and Loewe synergy scores in various cell lines treated with DS-1062 combined with AZD6738.

As seen from FIGS. 12A and 12B, and Table 2, AZD6738 interacted synergistically with DS-1062 and also increased cell death in TROP2-expressing lung cancer cell lines. As seen from FIGS. 13A and 13B, and Table 3, AZD6738 interacted synergistically with DS-1062 and also increased cell death in TROP2-expressing breast cancer cell lines at Emax (3 μM AZD6738 and 10 μg/ml DS-1062). As seen from FIG. 14, in eight cell lines, treatment with DS-1062 combined with AZD6738 resulted in high combination Emax (>100) and high Loewe synergy scores (>5).

The results in Example 3 demonstrate that ATR inhibition using AZD6738 enhances the antitumor efficacy of DS-1062 in TROP2-expressing lung and breast cancer cell lines in vitro. In Example 3, AZD6738 in combination with DS-1062 showed combination benefit in four TROP2-expressing lung cancer cell lines (FIGS. 12A, 12B, 14 and Table 2) and four TROP2-expressing breast cancer cell lines (FIGS. 13A, 13B and 14, and Table 3).

### Example 4: Antitumor Test—In Vivo—NCI-N87 Xenograft Model

Combination of antibody-drug conjugate DS-1062 (datopotamab deruxtecan) with ATR inhibitor AZD6738 (4-{4-[(3R)-3-Methylmorpholin-4-yl]-6-[1-((R)-S-methylsulfonimidoyl)cyclopropyl]pyrimidin-2-yl}-1H-pyrrolo[2,3-b]pyridine)

### Method:

Female Nude mice (Charles River) aged 5-8 weeks were used, following 7 days acclimatisation before entry into the study. 5×10⁶ NCI-N87 tumor cells (gastric cancer cell line) (1:1 in Matrigel) were implanted subcutaneously onto the flank of the female Nude mice. When tumors reached approximately 250 mm³, similar-sized tumors were randomly assigned to treatment groups as shown in Table 4:

TABLE 4

| | | Route of | Dosing Schedule |
|---|---|---|---|
| Treatment | Dose | administration | (14 days) |
| Vehicle | — | IV + PO | Single dose + QD |
| DS-1062 | 10 mg/kg | IV | Single dose |
| AZD6738 | 25 mg/kg | PO | QD |
| DS-1062 + | 10 mg/kg + | IV + PO | Single dose + QD |
| AZD6738 | 25 mg/kg | | |

PO: oral (per os) dosing
QD: once per day (quaque die) dosing

The dose of compound for each animal was calculated based on the individual body weight on the day of dosing. DS-1062 and AZD6738 were dosed on the same day, with DS-1062 being administered approximately 5 hour post the PO dose of AZD6738. DS-1062 was administered as a single dose at 10 mg/kg on day 1, and AZD6738 was administered at 25 mg/kg QD for 14 days. Duration of dosing was for 14 days.

Formulation of DS-1062 at 10 mg/kg

The dosing solutions for DS-1062 were prepared on the day of dosing by diluting the DS-1062 stock (20.1 mg/ml) in 25 mM histidine buffer, 9% sucrose (pH5.5) to 0.6 mg/ml, and 2 mg/ml for the 3 mg/kg and 10 mg/kg dosing solutions, respectively. Each dosing solution was mixed well using a pipette before administration via IV injection at a dosing volume of 5 ml/kg.

Formulation of AZD6738 at 25 mg/kg

To formulate for a 25 mg/kg dosing solution, a concentration of 2.5 mg/ml AZD6738 was prepared which resulted in a dosing volume of 10 ml/kg for PO dosing. DMSO (10% of the total vehicle volume) was added to the compound and mixed well with a pellet pestle. Sonication for approximately 5 minutes was required to fully dissolve the compound. Following which, propylene glycol (40% of the total vehicle volume) was added and mixed well using a magnetic stirrer. A volume of 10 ml of sterile water was added to the glass wheaton vial to rinse any remaining compound from the vial then transferred to the glass bottle. The remaining volume of sterile water in total (50% of the final vehicle volume) was added to the glass bottle and mixed well using a magnetic stirrer. The dosing solution was protected from light and kept at room temperature for up to 7 days being continually mixed. The final dosing matrix for 25 mg/kg AZD6738 was a clear solution with a faint yellow hue.

### Measurements

Tumor growth inhibition (TGI) was calculated as follows:

$$TGI \% = \{ 1 - ( MTV treated / MTV control ) \} ⋆ 100 \\ where MTV = mean tumor volume .$$

Statistical significance was evaluated using one-tailed t-test of (log(relative tumor volume)=log(final vol/start vol)) at the day of final measure, comparing to vehicle control.

### Results

Tumor volumes for treatments with DS-1062 or AZD6738 alone or with DS-1062 in combination with AZD6738 are shown in FIG. 15. Data represents change in tumor volume over time for treatment groups. The dotted line in FIG. 15 represents end of dosing periods. For full dose and schedule information, refer to Table 4 above. Values shown are geometric mean±SEM; n=8 for all treatment groups. The mice which had reached the humane endpoint (e.g. large tumor burden) during the study were removed from the group and the remaining mice were used for the calculation of the TGI.

TGI responses (Day 33 TGI %) following treatment with DS-1062 or AZD6738 alone or with DS-1062 in combination with AZD6738, in NCI-N87 xenograft, are shown in Table 5:

TABLE 5

| | | p-value | |
|---|---|---|---|
| | TGI % | vs | |
| Treatment group | day 33 | vehicle | Significance |
| DS-1062 10 mg/kg | 84.1 | <0.0001 | **** |
| AZD6738 25 mg/kg | 30.1 | 0.3316 | †ns |
| DS-1062 10 mg/kg + | 86.4 | <0.0001 | **** |
| AZD6738 25 mg/kg | | | |

†not significant

Monotherapy with DS-1062 at 10 mg/kg showed TGI value of 84.1% at day 33 post treatment. AZD6738 monotherapy achieved a TGI of 30.1% at day 33 post treatment. Combination treatment of AZD6738 with DS-1062 at 10 mg/kg resulted in a TGI of 86.4% at 33 days post treatment and showed better response than either respective monotherapies.

Treatment groups were generally well tolerated and average bodyweights of all treatment groups remained stable during the study.

### Example 5: Antitumor Test—In Vivo—TNBC Patient Derived Xenograft Model

Combination of antibody-drug conjugate DS-1062 (datopotamab deruxtecan) with ATR inhibitor AZD6738 (4-{4-[(3R)-3-Methylmorpholin-4-yl]-6-[1-((R)-S-methylsulfonimidoyl)cyclopropyl]pyrimidin-2-yl}-1H-pyrrolo[2,3-b]pyridine)

### Method:

Female Nude mice (Charles River) aged 5-8 weeks were used, following 7 days acclimatisation before entry into the study. The human patient-derived xenograft (PDX) model, CTG-3303, was established from fragments of freshly resected tumor of a triple negative breast cancer (TNBC) patient whom relapsed on treatment with PARP inhibitor talazoparib. This PDX was obtained in accordance with appropriate consent procedures. This TNBC PDX model was subcutaneously passaged in vivo as fragments from animal to animal in nude mice. When tumors reached approximately 250 mm³, similar-sized tumors were randomly assigned to treatment groups as shown in Table 6:

TABLE 6

| | | Route of | Dosing Schedule |
|---|---|---|---|
| Treatment | Dose | administration | (14 days) |
| Vehicle | — | IV + PO | Single dose + QD |
| DS-1062 | 10 mg/kg | IV | Single dose |
| AZD6738 | 25 mg/kg | PO | QD |
| DS-1062 + | 10 mg/kg + | IV + PO | Single dose + QD |
| AZD6738 | 25 mg/kg | | |

PO: oral (per os) dosing
QD: once per day (quaque die) dosing

The dose of compound for each animal was calculated based on the individual body weight on the day of dosing. DS-1062 and AZD6738 were dosed on the same day, with DS-1062 being administered approximately 5 hour post the PO dose of AZD6738. DS-1062 was administered as a single dose at 10 mg/kg on day 1, and AZD6738 was administered at 25 mg/kg QD for 14 days. Duration of dosing was for 14 days.

Formulation of DS-1062 at 10 mg/kg

The dosing solutions for DS-1062 were prepared on the day of dosing by diluting the DS-1062 stock (20.1 mg/ml) in 25 mM histidine buffer, 9% sucrose (pH5.5) to 0.6 mg/ml, and 2 mg/ml for the 3 mg/kg and 10 mg/kg dosing solutions, respectively. Each dosing solution was mixed well using a pipette before administration via IV injection at a dosing volume of 5 ml/kg.

Formulation of AZD6738 at 25 mg/kg

To formulate for a 25 mg/kg dosing solution, a concentration of 2.5 mg/ml AZD6738 was prepared which resulted in a dosing volume of 10 ml/kg for PO dosing. DMSO (10% of the total vehicle volume) was added to the compound and mixed well with a pellet pestle. Sonication for approximately 5 minutes was required to fully dissolve the compound. Following which, propylene glycol (40% of the total vehicle volume) was added and mixed well using a magnetic stirrer. A volume of 10 ml of sterile water was added to the glass wheaton vial to rinse any remaining compound from the vial then transferred to the glass bottle. The remaining volume of sterile water in total (50% of the final vehicle volume) was added to the glass bottle and mixed well using a magnetic stirrer. The dosing solution was protected from light and kept at room temperature for up to 7 days being continually mixed. The final dosing matrix for 25 mg/kg AZD6738 was a clear solution with a faint yellow hue.

### Results

Tumor volumes for treatments with DS-1062 or AZD6738 alone or with DS-1062 in combination with AZD6738 are shown in FIG. 16A. Data represents change in tumor volume over time for treatment groups. The dotted line in FIG. 16A represents end of dosing periods. For full dose and schedule information, refer to Table 6 above.

Values shown are geometric mean±SEM; n=8 for all treatment groups. The mice which had reached the humane endpoint (e.g. large tumor burden) during the study were removed from the group and the remaining mice were used for the calculation of the TGI.

TGI responses (Day 46 TGI %) following treatment with DS-1062 or AZD6738 alone or with DS-1062 in combination with AZD6738, in CTG-3303 xenograft, are shown in Table 7:

TABLE 7

| | | p-value | |
|---|---|---|---|
| | TGI % | vs | |
| Treatment group | day 46 | vehicle | Significance |
| DS-1062 10 mg/kg | 88.2 | <0.0001 | **** |
| AZD6738 25 mg/kg | 2.3 | 0.5112 | †ns |
| DS-1062 10 mg/kg + | 89.9 | <0.0001 | **** |
| AZD6738 25 mg/kg | | | |

Monotherapy with DS-1062 at 10 mg/kg showed TGI value of 88.2% at day 46 post treatment. AZD6738 monotherapy achieved a TGI of 2.3% at day 46 post treatment. Combination treatment of AZD6738 with DS-1062 at 10 mg/kg resulted in a TGI of 89.9% at 46 days post treatment and showed better response than either respective monotherapy.

Treatment groups were generally well tolerated and average bodyweights of all treatment groups remained stable during the study.

In addition, the percentage of mice in each treatment group achieving a complete response (defined as tumor volume=0 mm³) at day 93 post treatment were calculated and shown in FIG. 16B. Monotherapy with DS-1062 at 10 mg/kg led to 0 out 8 mice (0%) achieving a complete response at day 93 post treatment. AZD6738 monotherapy led to 0 out 8 mice (0%) achieving a complete response at day 93 post treatment. Combination treatment of AZD6738 with DS-1062 at 10 mg/kg led to 2 out 8 mice (25%) achieving a complete response at day 93 post treatment and led to higher complete response rates than either respective monotherapy.

### Example 6: Combination Dosing of Antibody-Drug Conjugate DS-1062 (Datopotamab Deruxtecan) with ATR Inhibitor AZD6738 in Hematopoietic Stem and Progenitors Cells In Vitro

### Method:

Cryopreserved human bone marrow derived CD34+ hematopoietic stem and progenitor cells (HSPCs; Lonza) were defrosted and left to recover overnight in maintenance media (StemSpan SFEM II (Stem Cell Technologies) containing 25 ng/ml SCF, 50 ng/ml TPO, and 50 ng/ml Flt3-L human recombinant protein (all Peprotech)), in a humidified incubator at 37° C. with 5% CO₂. The next day cells were resuspended in the presence of drug into media capable of supporting erythroid cell differentiation (Preferred Cell Systems, SEC-BFU1-40H), at a concentration of 10,000 cells/ml. Cells (30 μl) were plated into black-walled, clear bottomed 384 well tissue culture plates (Perkin Elmer) with the addition of 200, 70, 20, 8.3, 3.3 or 0 μg/ml DS-1062 (equivalent to 1.333, 0.467, 0.133, 0.056, 0.022 and 0 μM respectively) in combination with ATR inhibitor AZD6738 (1, 0.33, 0.167, 0.033, 0.017, 0 μM) in a 6×6 matrix pattern. Cells were cultured for 5 days in a humidified incubator at 37° C., with 5% CO₂.

Viability was determined using CellTiter-Glo 2.0 from Promega (using an optimised volume of 3 μl/well), with luminescence detected using an Envision plate reader (Perkin Elmer). Relative Luminescence signal was normalised in Genedata Screener software (Genedata) to percentage of control wells (0 μM of both compounds) with control wells equalling 0 and maximum cell death equalling 100. Synergy analysis was assessed using Loewe, Bliss, and Highest Single Agent (HSA) models with synergy scores and excess matrices determined by comparing the difference between the observed viability and that predicted based on a non-synergistic interaction for each combination dose pair.

### Results:

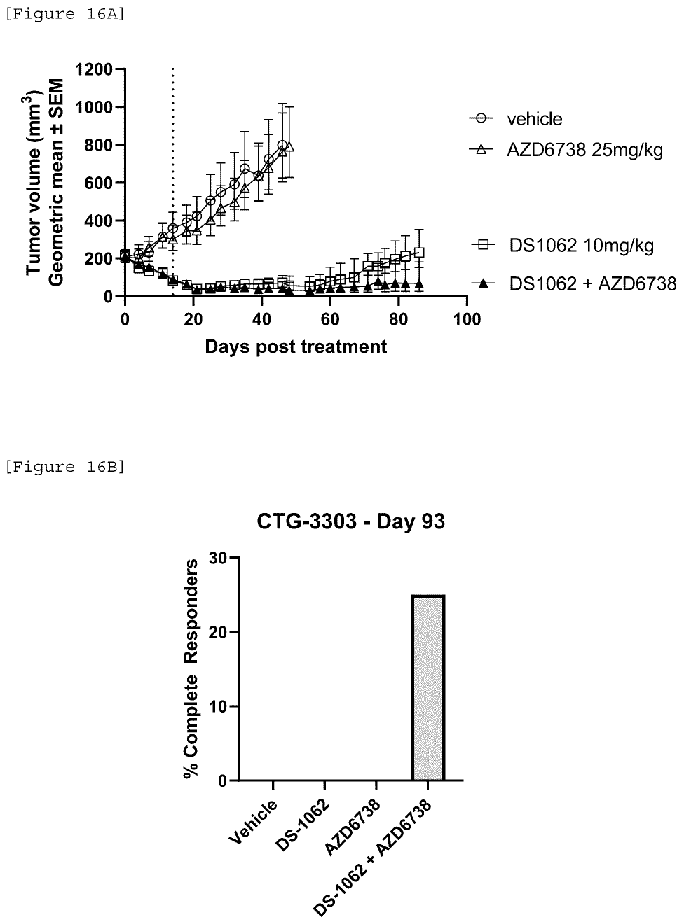

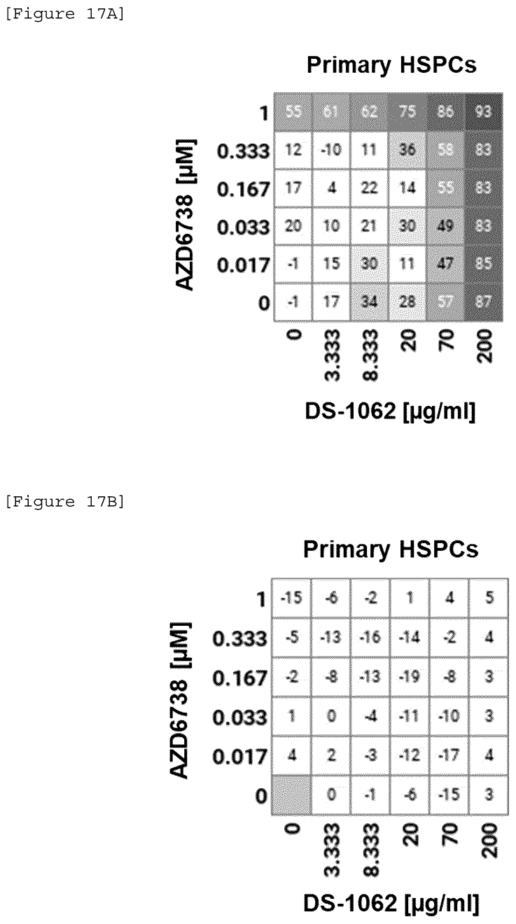

Results obtained with the combination dosings of DS-1062 with AZD6738 in primary CD34⁺ bone marrow-derived HSPCs induced to differentiate into the erythroid lineage are shown in FIGS. 17A and 17B and Table 8.

In FIG. 17A, measured cell viability signals are shown, with the X axis representing drug A (DS-1062) concentrations and the Y axis representing drug B (AZD6738) concentrations. Values in the boxes represent the % growth inhibition of cells treated with drug A+B, normalised to control values which equalled 0, with maximal cell death equalling 100. FIG. 17B shows the Loewe excess matrix, in which values in the boxes represent excess values calculated by the Loewe additivity model. Table 8 shows HSA synergy and Loewe additivity scores.

TABLE 8
DS-1062 and AZD6738 Combination Results in
Primary Hematopoietic Stem and Progenitor Cells

| Cell population | CD34+ HSPCs |
|---|---|
| HSA synergy score | 0.4 |
| Loewe synergy score | −0.2 |

There was no synergistic toxicity seen with concurrent DS-1062 and AZD6738 treatment in primary bone marrow derived CD34⁺ HSPCs differentiated into the erythroid lineage, with cell death in combination occurring at monotherapy active doses and following the predicted Loewe additivity interaction.

### Example 7: Antitumor Test—In Vivo—Dose Optimization-NCI-N87 Xenograft Model

Combination of antibody-drug conjugate DS-1062 (datopotamab deruxtecan) with ATR inhibitor AZD6738 (4-{4-[(3R)-3-Methylmorpholin-4-yl]-6-[1-((R)-S-methylsulfonimidoyl)cyclopropyl]pyrimidin-2-yl}-1H-pyrrolo[2,3-b]pyridine)

### Method:

Female Nude mice (Envigo) aged 5-8 weeks were used, following 7 days acclimatisation before entry into the study. 5×10⁶ NCI-N87 tumor cells (gastric cancer cell line) (1:1 in Matrigel) were implanted subcutaneously onto the flank of the female Nude mice. When tumors reached approximately 250 mm³, similar-sized tumors were randomly assigned to treatment groups as shown in Table 9:

TABLE 9

| | | Route of | |
|---|---|---|---|
| Treatment | Dose | administration | Dosing Schedule |
| Vehicle | | IV + PO | Single dose + |
| | | | Day 1-14 |
| DS-1062 | 10 mg/kg | IV | Single dose |
| AZD6738 | 25 mg/kg | PO | BID Day 1-14 |
| DS-1062 + | 10 mg/kg + | IV + PO | Single dose + BID |
| AZD6738 | 25 mg/kg | | Day 1-14 |
| DS-1062 + | 10 mg/kg + | IV + PO | Single dose + BID |
| AZD6738 | 25 mg/kg | | Day 1-7 |
| DS-1062 + | 10 mg/kg + | IV + PO | Single dose + BID |
| AZD6738 | 25 mg/kg | | Day 3-17 |
| DS-1062 + | 10 mg/kg + | IV + PO | Single dose + BID |
| AZD6738 | 25 mg/kg | | Day 7-21 |
| DS-1062 + | 10 mg/kg + | IV + PO | Single dose + BID |
| AZD6738 | 25 mg/kg | | Day 7-14 |

PO: oral (per os) dosing
BID: twice per day (bis in die) dosing

The dose of compound for each animal was calculated based on the individual body weight on the day of dosing. DS-1062 was administered as a single dose at 10 mg/kg on day 1, and AZD6738 was administered at 25 mg/kg BID as listed in Table 9.

Formulation of DS-1062 at 10 mg/kg

The dosing solution for DS-1062 was prepared on the day of dosing by diluting the DS-1062 stock (20.1 mg/ml) in 25 mM histidine buffer, 9% sucrose (pH5.5) to 2 mg/ml for 10 mg/kg dosing solution. Each dosing solution was mixed well using a pipette before administration via IV injection at a dosing volume of 5 ml/kg.

Formulation of AZD6738 at 25 mg/kg

To formulate for a 25 mg/kg dosing solution, a concentration of 2.5 mg/ml AZD6738 was prepared which resulted in a dosing volume of 10 ml/kg for PO dosing. DMSO (10% of the total vehicle volume) was added to the compound and mixed well with a pellet pestle. Sonication for approximately 5 minutes was required to fully dissolve the compound. Following which, propylene glycol (40% of the total vehicle volume) was added and mixed well using a magnetic stirrer. A volume of 10 ml of sterile water was added to the glass wheaton vial to rinse any remaining compound from the vial then transferred to the glass bottle. The remaining volume of sterile water in total (50% of the final vehicle volume) was added to the glass bottle and mixed well using a magnetic stirrer. The dosing solution was protected from light and kept at room temperature for up to 7 days being continually mixed. The final dosing matrix for 25 mg/kg AZD6738 was a clear solution with a faint yellow hue.

### Measurements

Tumor growth inhibition (TGI) was calculated as follows:

$$TGI \% = \{ 1 - ( MTV treated / MTV control ) \} ⋆ 100 \\ where MTV = mean tumor volume .$$

Statistical significance was evaluated using one-tailed t-test of (log(relative tumor volume)=log(final vol/start vol)) at the day of final measure, comparing to vehicle control.

### Results

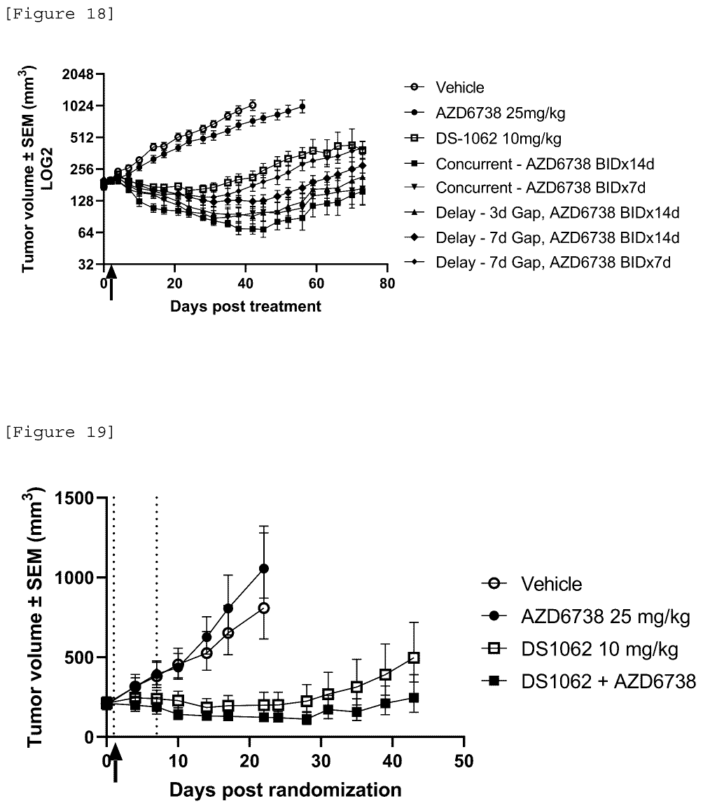

Tumor volumes for treatments with DS-1062 or AZD6738 alone or with DS-1062 in combination with AZD6738 are shown in FIG. 18. Data represents change in tumor volume over time for treatment groups. For full dose and schedule information, refer to Table 9 above. Values shown are geometric mean±SEM; n=8 for all treatment groups. The mice which had reached the humane endpoint (e.g. large tumor burden) during the study were removed from the group and the remaining mice were used for the calculation of the TGI.

TGI responses (Day 42 TGI %) following treatment with DS-1062 or AZD6738 alone or with DS-1062 in combination with AZD6738, in NCI-N87 xenograft, are shown in Table 10:

TABLE 10

| | | p-value | |
|---|---|---|---|
| | TGI % | vs | |
| Treatment group | day 42 | vehicle | Significance |
| DS-1062 10 mg/kg | 74.8 | <0.0001 | **** |
| AZD6738 25 mg/kg | 23.7 | 0.3519 | †ns |
| DS-1062 10 mg/kg + | 92.9 | <0.0001 | **** |
| AZD6738 25 mg/kg | | | |
| (Day 1-14) | | | |
| DS-1062 10 mg/kg + | 89.5 | <0.0001 | **** |
| AZD6738 25 mg/kg | | | |
| (Day 1-7) | | | |
| DS-1062 10 mg/kg + | 89.6 | <0.0001 | **** |
| AZD6738 25 mg/kg | | | |
| (Day 3-17) | | | |
| DS-1062 10 mg/kg + | 87.2 | <0.0001 | **** |
| AZD6738 25 mg/kg | | | |
| (Day 7-21) | | | |
| DS-1062 10 mg/kg + | 80.4 | <0.0001 | **** |
| AZD6738 25 mg/kg | | | |
| (Day 7-14) | | | |

†not significant

Monotherapy with DS-1062 at 10 mg/kg showed TGI value of 74.8% at day 42 post treatment. AZD6738 monotherapy achieved a TGI of 23.7% at day 42 post treatment. Combination treatment of AZD6738 with DS-1062 (Day1-14) resulted in a TGI of 92.9% at 42 days post treatment, Combination treatment of AZD6738 with DS-1062 (Day1-7) resulted in a TGI of 89.5% at 42 days post treatment, Combination treatment of AZD6738 with DS-1062 (Day3-17) resulted in a TGI of 89.6% at 42 days post treatment, Combination treatment of AZD6738 with DS-1062 (Day7-21) resulted in a TGI of 87.2% at 42 days post treatment and Combination treatment of AZD6738 with DS-1062 (Day7-14) resulted in a TGI of 80.4% at 42 days post treatment, indicating that a dose delay of AZD6738 following DS-1062 administration does not impact combination efficacy in N87 tumor model.

Treatment groups were generally well tolerated and average bodyweights of all treatment groups remained stable during the study.

### Example 8: Antitumor Test—In Vivo—CTG3718 Ovarian Patient Derived Xenograft Model

Combination of antibody-drug conjugate DS-1062 (datopotamab deruxtecan) with ATR inhibitor AZD6738 (4-{4-[(3R)-3-Methylmorpholin-4-yl]-6-[1-((R)-S-methylsulfonimidoyl)cyclopropyl]pyrimidin-2-yl}-1H-pyrrolo[2,3-b]pyridine)

### Method:

Female Nude mice (Envigo) aged 5-8 weeks were used, following 7 days acclimatisation before entry into the study. The human patient-derived xenograft (PDX) model, CTG-3718, was established from fragments of freshly resected tumor of a ovarian cancer patient whom relapsed on treatment with PARP inhibitor talazoparib. This PDX was obtained in accordance with appropriate consent procedures. This ovarian PDX model was subcutaneously passaged in vivo as fragments from animal to animal in nude mice. When tumors reached approximately 250 mm³, similar-sized tumors were randomly assigned to treatment groups as shown in Table 11:

TABLE 11

| | | Route of | |
|---|---|---|---|
| Treatment | Dose | administration | Dosing Schedule |
| Vehicle | — | IV + PO | Single dose + BID |
| | | | Day 1-14 |
| DS-1062 | 10 mg/kg | IV | Single dose |
| AZD6738 | 25 mg/kg | PO | BID Day 1-14 |
| DS-1062 + | 10 mg/kg + | IV + PO | Single dose + BID |
| AZD6738 | 25 mg/kg | | Day 1-14 |

PO: oral (per os) dosing
BID: twice per day (bis in die) dosing

The dose of compound for each animal was calculated based on the individual body weight on the day of dosing. DS-1062 and AZD6738 were dosed on the same day, with DS-1062 being administered approximately 5 hour post the PO dose of AZD6738. DS-1062 was administered as a single dose at 10 mg/kg on day 1, and AZD6738 was administered at 25 mg/kg BID for 14 days.

Formulation of DS-1062 at 10 mg/kg

The dosing solution for DS-1062 was prepared on the day of dosing by diluting the DS-1062 stock (20.1 mg/ml) in 25 mM histidine buffer, 9% sucrose (pH5.5) to 2 mg/ml for 10 mg/kg dosing solution. The dosing solution was mixed well using a pipette before administration via IV injection at a dosing volume of 5 ml/kg.

Formulation of AZD6738 at 25 mg/kg

To formulate for a 25 mg/kg dosing solution, a concentration of 2.5 mg/ml AZD6738 was prepared which resulted in a dosing volume of 10 ml/kg for PO dosing. DMSO (10% of the total vehicle volume) was added to the compound and mixed well with a pellet pestle. Sonication for approximately 5 minutes was required to fully dissolve the compound. Following which, propylene glycol (40% of the total vehicle volume) was added and mixed well using a magnetic stirrer. A volume of 10 ml of sterile water was added to the glass wheaton vial to rinse any remaining compound from the vial then transferred to the glass bottle. The remaining volume of sterile water in total (50% of the final vehicle volume) was added to the glass bottle and mixed well using a magnetic stirrer. The dosing solution was protected from light and kept at room temperature for up to 7 days being continually mixed. The final dosing matrix for 25 mg/kg AZD6738 was a clear solution with a faint yellow hue.

### Results

Tumor volumes for treatments with DS-1062 or AZD6738 alone or with DS-1062 in combination with AZD6738 are shown in FIG. 19. Data represents change in tumor volume over time for treatment groups. The dotted line in FIG. 19 represents end of dosing periods. For full dose and schedule information, refer to Table 11 above. Values shown are geometric mean±SEM; n=8 for all treatment groups. The mice which had reached the humane endpoint (e.g. large tumor burden) during the study were removed from the group and the remaining mice were used for the calculation of the TGI.

TGI responses (Day 22 TGI %) following treatment with DS-1062 or AZD6738 alone or with DS-1062 in combination with AZD6738, in CTG-3718 xenograft, are shown in Table 12:

TABLE 12

| | | p-value | |
|---|---|---|---|
| | TGI % | vs | |
| Treatment group | day 22 | vehicle | Significance |
| DS-1062 10 mg/kg | 69.8 | 0.007 | ** |
| AZD6738 25 mg/kg | −25.7 | >0.9999 | †ns |
| DS-1062 10 mg/kg + | 84.8 | 0.0012 | ** |
| AZD6738 25 mg/kg | | | |

Monotherapy with DS-1062 at 10 mg/kg showed TGI value of 69.8% at day 22 post treatment. AZD6738 monotherapy achieved a TGI of −25.7% at day 22 post treatment. Combination treatment of AZD6738 with DS-1062 at 10 mg/kg resulted in a TGI of 84.8% at 22 days post treatment and showed better response than either respective monotherapy.

Treatment groups were generally well tolerated and average bodyweights of all treatment groups remained stable during the study.

### Example 9: Antitumor Test—In Vivo—N87 (Gastric Cancer Cell Line)—DS-1062 Resistant Model

Combination of antibody-drug conjugate DS-1062 (datopotamab deruxtecan) with ATR inhibitor AZD6738 (4-{4-[(3R)-3-Methylmorpholin-4-yl]-6-[1-((R)-S-methylsulfonimidoyl)cyclopropyl]pyrimidin-2-yl}-1H-pyrrolo[2,3-b]pyridine)

### Method:

Female Nude mice (Charles River) aged 5-8 weeks were used, following 7 days acclimatisation before entry into the study. 5×10⁶ N87-DS-1062 resistant tumor cells (gastric cancer cell line) (1:1 in Matrigel) were implanted subcutaneously onto the flank of the female Nude mice. When tumors reached approximately 250 mm³, similar-sized tumors were randomly assigned to treatment groups as shown in Table 13:

TABLE 13

| | | Route of | |
|---|---|---|---|
| Treatment | Dose | administration | Dosing Schedule |
| Vehicle | — | IV + PO | Single dose + BID |
| | | | Day 1-7 |
| DS-1062 | 10 mg/kg | IV | Single dose |
| AZD6738 | 25 mg/kg | PO | BID Day 1-7 |
| DS-1062 + | 10 mg/kg + | IV + PO | Single dose + BID |
| AZD6738 | 25 mg/kg | | Day 1-7 |

PO: oral (per os) dosing
BID: twice per day (bis in die) dosing

The dose of compound for each animal was calculated based on the individual body weight on the day of dosing. DS-1062 and AZD6738 were dosed on the same day, with DS-1062 being administered approximately 5 hour post the PO dose of AZD6738. DS-1062 was administered as a single dose at 10 mg/kg on day 1, and AZD6738 was administered at 25 mg/kg BID for 7 days.

Formulation of DS-1062 at 10 mg/kg

The dosing solution for DS-1062 was prepared on the day of dosing by diluting the DS-1062 stock (20.1 mg/ml) in 25 mM histidine buffer, 9% sucrose (pH5.5) to 2 mg/ml for 10 mg/kg dosing solution. The dosing solution was mixed well using a pipette before administration via IV injection at a dosing volume of 5 ml/kg.

Formulation of AZD6738 at 25 mg/kg

To formulate for a 25 mg/kg dosing solution, a concentration of 2.5 mg/ml AZD6738 was prepared which resulted in a dosing volume of 10 ml/kg for PO dosing. DMSO (10% of the total vehicle volume) was added to the compound and mixed well with a pellet pestle. Sonication for approximately 5 minutes was required to fully dissolve the compound. Following which, propylene glycol (40% of the total vehicle volume) was added and mixed well using a magnetic stirrer. A volume of 10 ml of sterile water was added to the glass wheaton vial to rinse any remaining compound from the vial then transferred to the glass bottle. The remaining volume of sterile water in total (50% of the final vehicle volume) was added to the glass bottle and mixed well using a magnetic stirrer. The dosing solution was protected from light and kept at room temperature for up to 7 days being continually mixed. The final dosing matrix for 25 mg/kg AZD6738 was a clear solution with a faint yellow hue.

### Results

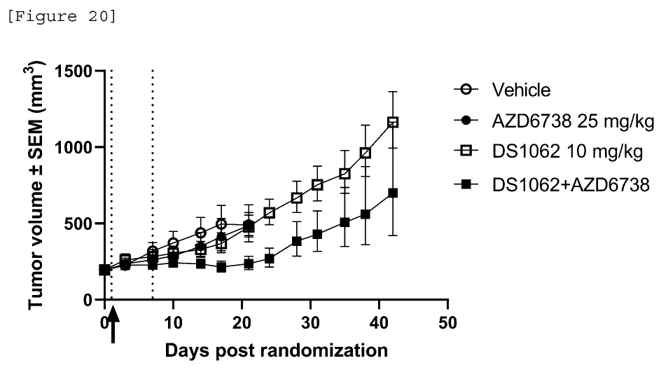

Tumor volumes for treatments with DS-1062 or AZD6738 alone or with DS-1062 in combination with AZD6738 are shown in FIG. 20. Data represents change in tumor volume over time for treatment groups. The dotted line in FIG. 20 represents end of dosing periods. For full dose and schedule information, refer to Table 13 above. Values shown are geometric mean±SEM; n=8 for all treatment groups. The mice which had reached the humane endpoint (e.g. large tumor burden) during the study were removed from the group and the remaining mice were used for the calculation of the TGI.

TGI responses (Day 21 TGI %) following treatment with DS-1062 or AZD6738 alone or with DS-1062 in combination with AZD6738, in N87-DS-1062 resistant tumor cell xenograft, are shown in Table 14:

TABLE 14

| | | p-value | |
|---|---|---|---|
| | TGI % | vs | |
| Treatment group | day 21 | vehicle | Significance |
| DS-1062 10 mg/kg | 0.77 | 0.3829 | †ns |
| AZD6738 25 mg/kg | −9.8 | 0.4557 | †ns |
| DS-1062 10 mg/kg + | 49.5 | 0.0728 | †ns |
| AZD6738 25 mg/kg | | | |

Monotherapy with DS-1062 at 10 mg/kg showed TGI value of 0.77% at day 21 post treatment. AZD6738 monotherapy achieved a TGI of −9.8% at day 21 post treatment. Combination treatment of AZD6738 with DS-1062 at 10 mg/kg resulted in a TGI of 49.5% at 21 days post treatment and showed better response than either respective monotherapy. These data suggest ATR inhibition, demonstrated herein with AZD6738, can re-senstizize DS-1062 resistant tumors to DS-1062 treatment.

Treatment groups were generally well tolerated and average bodyweights of all treatment groups remained stable during the study.

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiments may be practiced in many ways and the claims include any equivalents thereof.

### Free Text of Sequence Listing

SEQ ID NO: 1—Amino acid sequence of a heavy chain of anti-TROP2 antibody
SEQ ID NO: 2—Amino acid sequence of a light chain of anti-TROP2 antibody
SEQ ID NO: 3—Amino acid sequence of a heavy chain CDRH1 [=amino acid residues 50 to 54 of SEQ ID NO: 1]
SEQ ID NO: 4—Amino acid sequence of a heavy chain CDRH2 [=amino acid residues 69 to 85 of SEQ ID NO: 1]
SEQ ID NO: 5—Amino acid sequence of a heavy chain CDRH3 [=amino acid residues 118 to 129 of SEQ ID NO: 1]
SEQ ID NO: 6—Amino acid sequence of a light chain CDRL1 [=amino acid residues 44 to 54 of SEQ ID NO: 2]
SEQ ID NO: 7—Amino acid sequence of a light chain CDRL2 [=amino acid residues 70 to 76 of SEQ ID NO: 2]
SEQ ID NO: 8—Amino acid sequence of a light chain CDRL3 [=amino acid residues 109 to 117 of SEQ ID NO: 2]
SEQ ID NO: 9—Amino acid sequence of a heavy chain variable region [=amino acid residues 20 to 140 of SEQ ID NO: 1]
SEQ ID NO: 10—Amino acid sequence of a light chain variable region [=amino acid residues 21 to 129 of SEQ ID NO: 2]
SEQ ID NO: 11—Amino acid sequence of a heavy chain [=amino acid residues 20 to 469 of SEQ ID NO: 1]
SEQ ID NO: 12—Amino acid sequence of a heavy chain [=amino acid residues 20 to 470 of SEQ ID NO: 1]
SEQ ID NO: 13—Amino acid sequence of a light chain [=amino acid residues 21 to 234 of SEQ ID NO: 2]

1. A pharmaceutical product comprising an anti-TROP2 antibody-drug conjugate and an ATR inhibitor for administration in combination, wherein the anti-TROP2 antibody-drug conjugate is an antibody-drug conjugate in which a drug-linker represented by the following formula: 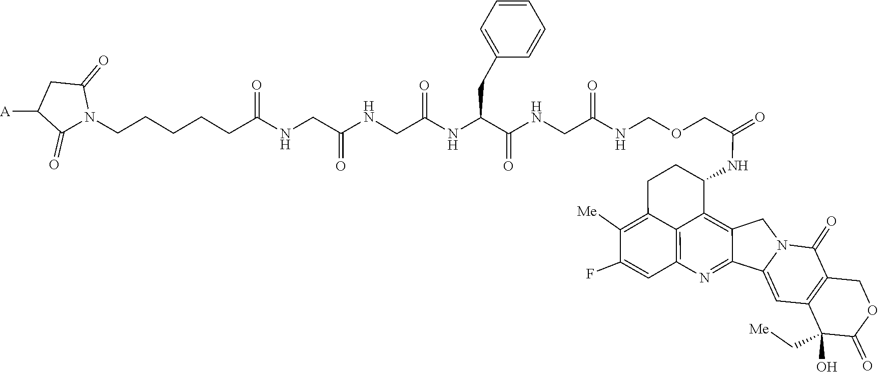
wherein A represents the connecting position to an antibody, is conjugated to an anti-TROP2 antibody via a thioether bond.

2. The pharmaceutical product according to claim 1, wherein the ATR inhibitor is a compound represented by the following formula (I): 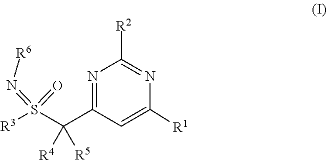
wherein:
R¹ is selected from morpholin-4-yl and 3-methylmorpholin-4-yl;
R² is 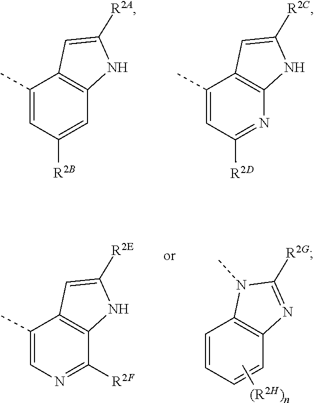
n is 0 or 1;
R^{2A}, R^{2C}, R^{2E} and R^{2F} each independently are hydrogen or methyl;
R^{2B} and R^{2D} each independently are hydrogen or methyl;
R^{2G} is selected from —NHR⁷ and —NHCOR⁸;
R^{2H} is fluoro;
R³ is methyl;
R⁴ and R⁵ are each independently hydrogen or methyl, or R⁴ and R⁵ together with the atom to which they are attached form Ring A;
Ring A is a C₃₋₆cycloalkyl or a saturated 4-6 membered heterocyclic ring containing one heteroatom selected from O and N;
R⁶ is hydrogen;
R⁷ is hydrogen or methyl;
R⁸ is methyl,
or a pharmaceutically acceptable salt thereof.

3. The pharmaceutical product according to claim 2 wherein, in formula (I), R⁴ and R⁵ together with the atom to which they are attached form Ring A, and Ring A is a C₃-6cycloalkyl or a saturated 4-6 heterocyclic ring containing one heteroatom selected from O and N.

4. The pharmaceutical product according to claim 2 or claim 3 wherein, in formula (I), Ring A is a cyclopropyl, tetrahydropyranyl or piperidinyl ring.

5. The pharmaceutical product according to any one of claims 2 to 4 wherein, in formula (I), R^{2A} is hydrogen; R^{2B} is hydrogen; R^{2C} is hydrogen; R^{2D} is hydrogen; R^{2E} is hydrogen; and R^{2F} is hydrogen.

6. The pharmaceutical product according to any one of claims 2 to 5 wherein, in formula (I), R¹ is 3-methylmorpholin-4-yl.

7. The pharmaceutical product according to any one of claims 2 to 6 wherein the compound of formula (I) is a compound of formula (Ia): 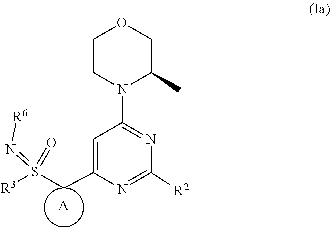
or a pharmaceutically acceptable salt thereof.

8. The pharmaceutical product according to claim 7 wherein, in formula (Ia):
Ring A is cyclopropyl ring;
R² is 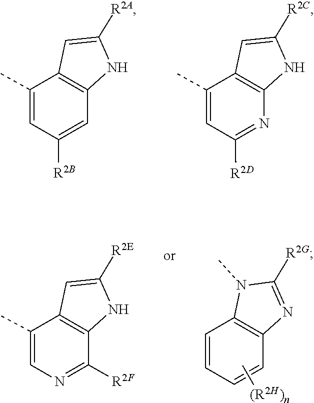
n is 0 or 1;
R^{2A} is hydrogen;
R^{2B} is hydrogen;
R^{2C} is hydrogen;
R^{2D} is hydrogen;
R^{2E} is hydrogen;
R^{2F} is hydrogen;
R^{2G} is —NHR⁷;
R^{2H} is fluoro;
R³ is a methyl group;
R⁶ is hydrogen; and
R⁷ is hydrogen or methyl.

9. The pharmaceutical product according to claim 2, wherein the ATR inhibitor is AZD6738 represented by the following formula: 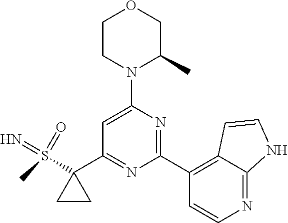 [SMILES: C[C@@H]1COCCN1c1cc(C2([S@](C)(=N)=O)CC2)nc(-c2ccnc3[nH]ccc23)n1]
or a pharmaceutically acceptable salt thereof.

10. The pharmaceutical product according to any one of claims 1 to 9, wherein the anti-TROP2 antibody is an antibody comprising a heavy chain comprising CDRH1 consisting of an amino acid sequence represented by SEQ ID NO: 3, CDRH2 consisting of an amino acid sequence represented by SEQ ID NO: 4 and CDRH3 consisting of an amino acid sequence represented by SEQ ID NO: 5, and a light chain comprising CDRL1 consisting of an amino acid sequence represented by SEQ ID NO: 6, CDRL2 consisting of an amino acid sequence represented by SEQ ID NO: 7 and CDRL3 consisting of an amino acid sequence represented by SEQ ID NO: 8.

11. The pharmaceutical product according to claim 10, wherein the anti-TROP2 antibody is an antibody comprising a heavy chain comprising a heavy chain variable region consisting of an amino acid sequence represented by SEQ ID NO: 9 and a light chain comprising a light chain variable region consisting of an amino acid sequence represented by SEQ ID NO: 10.

12. The pharmaceutical product according to claim 11, wherein the anti-TROP2 antibody is an antibody comprising a heavy chain consisting of an amino acid sequence represented by SEQ ID NO: 12 and a light chain consisting of an amino acid sequence represented by SEQ ID NO: 13.

13. The pharmaceutical product according to claim 11, wherein the anti-TROP2 antibody is an antibody comprising a heavy chain consisting of an amino acid sequence represented by SEQ ID NO: 11 and a light chain consisting of an amino acid sequence represented by SEQ ID NO: 13.

14. The pharmaceutical product according to any one of claims 1 to 13, wherein the average number of units of the drug-linker conjugated per antibody molecule in the antibody-drug conjugate is in the range of from 2 to 8.

15. The pharmaceutical product according to claim 14, wherein the average number of units of the drug-linker conjugated per antibody molecule in the antibody-drug conjugate is in the range of from 3.5 to 4.5.

16. The pharmaceutical product according to claim 15, wherein the anti-TROP2 antibody-drug conjugate is datopotamab deruxtecan (DS-1062).

17. The pharmaceutical product according to any one of claims 1 to 16 wherein the product is a combined preparation comprising the anti-TROP2 antibody-drug conjugate and the ATR inhibitor, for separate simultaneous administration.

18. The pharmaceutical product according to any one of claims 1 to 16 wherein the product is a combined preparation comprising the anti-TROP2 antibody-drug conjugate and the ATR inhibitor, for sequential or separate simultaneous administration.

19. The pharmaceutical product according to any one of claims 1 to 18 wherein the anti-TROP2 antibody-drug conjugate is to be administered at a dose of 6 mg/kg body weight.

20. The pharmaceutical product according to claim 19 wherein the dose of the anti-TROP2 antibody-drug conjugate is to be administered once every three weeks.

21. The pharmaceutical product according to any one of claims 1 to 20 wherein the ATR inhibitor is to be administered daily for the first week, second week, and/or third week of a three week cycle.

22. The pharmaceutical product according to any one of claims 1 to 20 wherein the ATR inhibitor is to be administered on days 3 to 17 of a three week cycle.

23. The pharmaceutical product according to any one of claims 1 to 22, wherein the product is for treating cancer.

24. The pharmaceutical product according to claim 23, wherein the cancer is at least one selected from the group consisting of breast cancer, lung cancer, colorectal cancer, gastric cancer, esophageal cancer, head-and-neck cancer, esophagogastric junction adenocarcinoma, biliary tract cancer, Paget's disease, pancreatic cancer, ovarian cancer, uterine carcinosarcoma, urothelial cancer, prostate cancer, bladder cancer, gastrointestinal stromal tumor, digestive tract stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma.

25. The pharmaceutical product according to claim 24, wherein the cancer is breast cancer.

26. The pharmaceutical product according to claim 25, wherein the breast cancer is triple negative breast cancer.

27. The pharmaceutical product according to claim 26, wherein the breast cancer is hormone receptor (HR)-positive, HER2-negative breast cancer.

28. The pharmaceutical product according to claim 27, wherein the cancer is lung cancer.

29. The pharmaceutical product according to claim 28, wherein the lung cancer is non-small cell lung cancer.

30. The pharmaceutical product according to claim 29, wherein the non-small cell lung cancer is non-small cell lung cancer with actionable genomic alterations.

31. The pharmaceutical product according to claim 30, wherein the non-small cell lung cancer is non-small cell lung cancer lung cancer without actionable genomic alterations.

32. The pharmaceutical product according to claim 24, wherein the cancer is colorectal cancer.

33. The pharmaceutical product according to claim 24, wherein the cancer is gastric cancer.

34. The pharmaceutical product according to claim 24, wherein the cancer is pancreatic cancer.

35. The pharmaceutical product according to claim 24, wherein the cancer is ovarian cancer.

36. The pharmaceutical product according to claim 24, wherein the cancer is prostate cancer.

37. The pharmaceutical product according to claim 24, wherein the cancer is kidney cancer.

38. The pharmaceutical product according to any one of claims 24 to 37, wherein the cancer is deficient in Homologous Recombination (HR) dependent DNA DSB repair activity.

39. The pharmaceutical product according to any one of claims 24 to 37, wherein the cancer is not deficient in Homologous Recombination (HR) dependent DNA DSB repair activity.

40. The pharmaceutical product according to any one of claims 24 to 37, wherein the cancer is exhibits resistance or refractoriness to a previous treatment with a PARP inhibitor.

41. The pharmaceutical product according to claim 40, wherein the previous treatment is with a PARP inhibitor selected from olaparib, rucaparib, niraparib, talazoparib and veliparib.

42. The pharmaceutical product according to any one of claims 24 to 37, wherein cancer cells of the cancer are SLFN11-deficient.

43. The pharmaceutical product according to claim 42, wherein SLFN11 expression is lower in the cancer cells of a patient relative to the patient's SLFN11-expressing non-cancer cells.

44. A pharmaceutical product as defined in any one of claims 1 to 22, for use in treating cancer.

45. The pharmaceutical product for the use according to claim 44, wherein the cancer is as defined in any one of claims 24 to 43.

46. Use of an anti-TROP2 antibody-drug conjugate in the manufacture of a medicament for use in combination with an ATR inhibitor, wherein the anti-TROP2 antibody-drug conjugate and the ATR inhibitor are as defined in any one of claims 1 to 16, for treating cancer.

47. The use according to claim 46 wherein the medicament is for use in combination with the ATR inhibitor by sequential administration.

48. The use according to claim 46 wherein the medicament is for use in combination with the ATR inhibitor by separate simultaneous administration.

49. Use of an ATR inhibitor in the manufacture of a medicament for use in combination with an anti-TROP2 antibody-drug conjugate, wherein the anti-TROP2 antibody-drug conjugate and the ATR inhibitor are as defined in any one of claims 1 to 16, for treating cancer.

50. The use according to claim 49 wherein the medicament is for use in combination with the anti-TROP2 antibody-drug conjugate by sequential administration.

51. The use according to claim 49 wherein the medicament is for use in combination with the anti-TROP2 antibody-drug conjugate by separate simultaneous administration.

52. The use according to any one of claims 46 to 51, wherein the cancer is as defined in any one of claims 24 to 43.

53. An anti-TROP2 antibody-drug conjugate for use, in combination with an ATR inhibitor, in the treatment of cancer, wherein the anti-TROP2 antibody-drug conjugate and the ATR inhibitor are as defined in any one of claims 1 to 16.

54. The anti-TROP2 antibody-drug conjugate for the use according to claim 53, wherein the cancer is as defined in any one of claims 24 to 43.

55. The anti-TROP2 antibody-drug conjugate for the use according to claim 53 or 54, wherein the use comprises administration of the anti-TROP2 antibody-drug conjugate and the ATR inhibitor sequentially.

56. The anti-TROP2 antibody-drug conjugate for the use according to claim 53 or 54, wherein the use comprises administration of the anti-TROP2 antibody-drug conjugate and the ATR inhibitor separately and simultaneously.

57. An anti-TROP2 antibody-drug conjugate for use in the treatment of cancer in a subject, wherein said treatment comprises the sequential or separate simultaneous administration of i) said anti-TROP2 antibody-drug conjugate, and ii) an ATR inhibitor to said subject, wherein said anti-TROP2 antibody-drug conjugate and said ATR inhibitor are as defined in any one of claims 1 to 16.

58. The anti-TROP2 antibody-drug conjugate for the use according to any one of claims 53 to 57 wherein the anti-TROP2 antibody-drug conjugate is to be administered at a dose of 6 mg/kg body weight.

59. The anti-TROP2 antibody-drug conjugate for the use according to claim 58 wherein the dose of the anti-TROP2 antibody-drug conjugate is to be administered once every three weeks.

60. The anti-TROP2 antibody-drug conjugate for the use according to any one of claims 53 to 59 wherein the ATR inhibitor is to be administered daily for the first week, second week, and/or third week of a three week cycle.

61. The anti-TROP2 antibody-drug conjugate for the use according to any one of claims 53 to 59 wherein the ATR inhibitor is to be administered on days 3 to 17 of a three week cycle.

62. An ATR inhibitor for use, in combination with an anti-TROP2 antibody-drug conjugate, in the treatment of cancer, wherein the anti-TROP2 antibody-drug conjugate and the ATR inhibitor are as defined in any one of claims 1 to 16.

63. The ATR inhibitor for the use according to claim 62, wherein the cancer is as defined in any one of claims 24 to 43.

64. The ATR inhibitor for the use according to claim 62 or 63, wherein the use comprises administration of the anti-TROP2 antibody-drug conjugate and the ATR inhibitor sequentially.

65. The ATR inhibitor for the use according to claim 62 or 63, wherein the use comprises administration of the anti-TROP2 antibody-drug conjugate and the ATR inhibitor separately and simultaneously.

66. An ATR inhibitor for use in the treatment of cancer in a subject, wherein said treatment comprises the sequential or separate simultaneous administration of i) said ATR inhibitor, and ii) an anti-TROP2 antibody-drug conjugate to said subject, wherein said ATR inhibitor and said anti-TROP2 antibody-drug conjugate are as defined in any one of claims 1 to 16.

67. The ATR inhibitor for the use according to any one of claims 62 to 66 wherein the anti-TROP2 antibody-drug conjugate is to be administered at a dose of 6 mg/kg body weight.

68. The ATR inhibitor for the use according to claim 67 wherein the dose of the anti-TROP2 antibody-drug conjugate is to be administered once every three weeks.

69. The ATR inhibitor for the use according to any one of claims 62 to 68 wherein the ATR inhibitor is to be administered daily for the first week, second week, and/or third week of a three week cycle.

70. The ATR inhibitor for the use according to any one of claims 62 to 68 wherein the ATR inhibitor is to be administered on days 3 to 17 of a three week cycle.

71. A method of treating cancer comprising administering an anti-TROP2 antibody-drug conjugate and an ATR inhibitor as defined in any one of claims 1 to 16 in combination to a subject in need thereof.

72. The method according to claim 71, wherein the cancer is as defined in any one of claims 24 to 43.

73. The method according to claim 71 or 72, wherein the method comprises administering the anti-TROP2 antibody-drug conjugate and the ATR inhibitor sequentially.

74. The method according to claim 71 or 72, wherein the method comprises administering the anti-TROP2 antibody-drug conjugate and the ATR inhibitor separately and simultaneously.


SEQUENCE LISTING (13 sequences)

SEQ ID NO: 1 - protein, 470 aa, synthetic construct
  REGION 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, synthetic construct
  REGION 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, synthetic construct
  REGION 1..5: note = CDRH1 of heavy chain of anti-TROP2 antibody
TAGMQ

SEQ ID NO: 4 - protein, 17 aa, synthetic construct
  REGION 1..17: note = CDRH2 of heavy chain of anti-TROP2 antibody
WINTHSGVPKYAEDFKG

SEQ ID NO: 5 - protein, 12 aa, synthetic construct
  REGION 1..12: note = CDRH3 of heavy chain of anti-TROP2 antibody
SGFGSSYWYFDV

SEQ ID NO: 6 - protein, 11 aa, synthetic construct
  REGION 1..11: note = CDRL1 of light chain of anti-TROP2 antibody
KASQDVSTAVA

SEQ ID NO: 7 - protein, 7 aa, synthetic construct
  REGION 1..7: note = CDRL2 of light chain of anti-TROP2 antibody
SASYRYT

SEQ ID NO: 8 - protein, 9 aa, synthetic construct
  REGION 1..9: note = CDRL3 of light chain of anti-TROP2 antibody
QQHYITPLT

SEQ ID NO: 9 - protein, 121 aa, synthetic construct
  REGION 1..121: note = Variable region of heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 10 - protein, 109 aa, synthetic construct
  REGION 1..109: note = Variable region of light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 11 - protein, 450 aa, synthetic construct
  REGION 1..450: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 451 aa, synthetic construct
  REGION 1..451: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = Light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC